(19) United States
(12) Patent Application Publication
(10) Pub. No.: US 2005/0244823 A1
(43) Pub. Date: Nov. 3, 2005

(54) Novel epitopes for celiac disease and autoimmune diseases, methods for detecting those and novel non-antigenic food compounds

(75) Inventors: Jan Wouter Drijfhout, Leiden (NL); Petrus Antonius van Veelen, Wassenaar (NL); Frits Koning, Leiderdorp (NL)

Correspondence Address: Cooper & Dunham, 1185 Avenue of the Americas, New York, NY, 10036, US

(73) Assignee: Academisch Ziekenhuis Leiden

(21) Appl. No.: 10/503,575

(22) PCT Filed: Feb. 4, 2003
(86) PCT No.: PCT/NL03/00077

§ 371 Date: Oct. 29, 2004

(30) Foreign Application Priority Data
Feb. 4, 2002 (EP) 02075456.0

(51) Int. Cl. C12Q 1/68; G01N 33/53; G01N 33/567; C07K 14/415; C07K 16/18

(52) U.S. Cl. 435/6; 530/324; 530/325; 530/326; 530/327; 435/7.2; 435/320.1; 435/325; 530/388.1; 536/23.1

(57) ABSTRACT

The invention describes the patterns of deamidation in gluten, and it is found that this is highly dependent on the spacing between the glutamine and proline residues. This knowledge can be used to predict novel T cell stimulatory gluten peptides. Several newly defined peptides and epitopes are provided. Also, the finding can explain the formation of neo-epitopes in autoimmune diseases such as RA (rheumatoid arthritis), MS (multiple sclerosis), SLE (systemic lupus erythomatosus), SS (Sjogren syndrome) and DB (diabetes). Several neo-epitopes and the peptides that are substrate for deamidation are provided. Further, the inventions provides for methods for detecting these peptides and epitopes and methods for making food more suitable for celiac disease patients.

The invention relates to the field of molecular biology and immunology, more specific to immune diseases, especially celiac disease. It further relates to neo-epitopes that can be generated by the enzyme tissue transglutaminase (tTG) which neo-epitopes can play a role in celiac disease and in autoimmune diseases.

### INTRODUCTION

Celiac disease (CD) is a permanent intolerance to gluten (Marsh, M. N., Gastroent. 102: 330-354, 1992). Gluten is a complex mixture of proteins found in wheat and several other grains, and is present in many food products. Typical disease symptoms include chronic diarrhoea, fatigue, and failure to thrive. These symptoms are associated with a lesion in the upper small intestine, characterised by a (sub) total villous atrophy, an increased number of intra epithelial lymphocytes, and a chronic inflammatory response of the lamina propria lymphocytes (Marsh, 1992). Diagnosis is based on morphology of a small intestinal biopsy and improvement of the clinical status after a gluten free diet. A strong indication for CD is the presence of antibodies specific for the enzyme tissue transglutaminase (tTG) in patients (Dieterich, W. et al., Nat. Med. 3: 797-801, 1997). Moreover, recent studies demonstrated the involvement of this enzyme in the generation of T cell stimulatory peptides (Molberg, O. et al., Nat. Med. 4: 713-717, 1998; van de Wal, Y. et al., J. Immunol. 161: 1585-1588, 1998a). CD is strongly associated with HLA-DQ2 (A1*0501, B1*0201) and HLA-DQ8 (A1*0301, B1*0302) (Spurkland A. et al., Tissue Antigens 4: 29-34, 1997). In addition, HLA-DQ2 and/or HLA-DQ8 restricted, gluten specific CD4⁺ T lymphocytes can be isolated from small intestinal biopsies of CD patients, and these are thought to cause disease (Lundin, K. et al., J. Exp. Med. 178: 187-196, 1993; van de Wal, Y. et al. Proc. Natl. Acad. Sci. USA 95: 10050-10054, 1998b). The peptide binding motif of HLA-DQ2 and -DQ8 predicts a preference for negative charges at anchor positions in the bound peptides (Johansen, B. H. et al., Int. Immunol. 8: 177-182, 1996; van de Wal, Y. et al. Immunogenetics 44: 246-253, 1996; Kwok, W. W. et al., J. Immunol. 156: 2171-2177, 1996). Gluten molecules, however, contain few negative charges. This discrepancy was solved by the finding that tTG can convert glutamine residues into glutamic acid (a process termed deamidation), and thus introduces negative charges in gluten peptides. Indeed it was found that tTG is required for, or enhances the gluten specific response of T cell clones and T cell lines derived from the majority of CD patients (van de Wal, 1998a; Molberg, 1998; Arentz-Hansen, H. et al., J. Exp. Med. 191: 603-612, 2000; Anderson, R. P. et al., Nat. Med. 6: 387-342, 2000). Considering the abundance of glutamine residues in gluten (˜30-40%) tTG has many potential target sites in gluten. Mass spectral analysis of the deamidated T cell stimulatory gluten peptides, however, showed a highly restricted pattern of deamidation (van de Wal, 1998a; Molberg, 1998). Usually the pattern of deamidation coincides with the positions where negative charges are preferred in the HLA binding motif, thus favouring the binding of gluten peptides to HLA-DQ2 and/or -DQ8. The specificity of gluten deamidation by tTG, therefore, is a crucial factor in the generation of toxic gluten peptides. It is not known, however, why only particular glutamine residues are targeted by tTG.

Current therapy of CD mainly involves dietary treatment of glutensensitive patients with diets lacking cereal compounds such as flour, which deprives these patients of such typical staple foods as for instance bread. The criteria for glutenfree products are established by the Codex Alimentarius Committe “Nutrition and Food for Special Dietary Uses” that meets every 1.5 year. The current criterion is based on the determination of nitrogen. To be considered glutenfree a product may contain maximally 50 mg N per 100 gram of product. The determination is only useful when there is a certain relationship between the amount of nitrogen and the amount of gluten. This is only true, to a certain extent, for wheat. There is still a need for a more reliable method to detect whether food materials would cause problems in CD patients.

For auto-immune diseases in general, it is not known which combination of factors gives rise to an immunological response. One of the hypotheses is that presumed antigenic compounds are processed in the human body and give rise to so-called neo-epitopes. These neo-epitopes are then recognised by the immune system and disease occurs. It is, however, not yet clear in which way these neo-epitopes can be formed.

### SUMMARY OF THE INVENTION

The invention now provides a peptide sequence of 14-40 amino acids, which is prone to deamidation by tTG and which is a causative factor of celiac disease, which comprises an amino acid sequence selected from the group consisting essentially of QQPYPQQPQQPFPQ, QQPFPQQPQQPFPQ, PFPQTQQPQQPFPQ, PFPQLQQPQQPFPQ, QFPQTQQPQQPFPQ, QLPFPQQPQQPFPQ, PFPQPQQPQQPFPQ and PFPQSQQPQQPFPQ. Also, the invention provides for epitopes made by deamidation of these peptides, thus resulting in the sequences QQPYPEQPQQPFPQ, QQPFPEQPQQPFPQ, PFPQTEQPQQPFPQ, PFPQLEQPQQPFPQ, QFPQTEQPQQPFPQ, QLPFPBQPQQPFPQ, PFPQPEQPQQPFPQ, PFPQSEQPQQPFPQ, QQPYPEQPEQPFPQ, QQPFPEQPEQPFPQ, PFPQTEQPEQPFPQ, PFPQLEQPEQPFPQ, QFPQTEQPEQPFPQ, QLPFPEQPFQPFPQ, PFPQPEQPEQPFPQ, PFPQSEQPEQPFPQ, QQPYPQQPEQPFPQ, QQPFPQQPEQPFPQ, PFPQTQQPEQPFPQ, PFPQLQQPEQPFPQ, QFPQTQQPEQPFPQ, QLPFPQQPEQPFPQ, PFPQPQQPEQPFPQ and PFPQSQQPEQPFPQ, wherein further Q residues may be deamidated into an E residue.

The invention also provides or peptide sequences, which are prone to deamidation by tTG and which are a causative factor of an autoimmune disease selected from the group consisting essentially of RA (rheumatoid arthritis), MS (multiple sclerosis), SLE (systemic lupus erythomatosus), SS (Sjogren syndrome) and DB (diabetes), which peptides are shown in table 5. Preferably these peptide sequences are residing in sequences 8-40 amino acid residues which comprise at least one 8-mer which is a subsequence of at least one of the sequences listed in claim 3, wherein said 8-mer comprises the central Q residue of the sequence listed in table 5. The invention also harbours the epitope formed by deamidation of one or more of the Q residues of these peptides.

A further part of the invention is an isolated HLA-DQ restricted T-cell capable of recognising a gluten-derived epitope according to the invention and more specifically an isolated or recombinant HLA-DQ restricted T-cell receptor capable of recognising such an epitope.

Also encompassed are antibodies reactive with a gluten-derived peptide or epitope according to the invention.

Further disclosed in the invention is a method to screen foodstuffs or plant material for the occurrence of these peptides or epitopes using such a T cell, a T cell receptor or antibody, Preferably in such a method the foodstuff or plant material is pretreated with a protease such as trypsin, chymotrypsin or pepsin. Most preferably in such a method the foodstuff or plant material is additionally treated with tTG.

Furthermore part of the invention is a diagnostic kit comprising: a T cell, a T cell receptor or an antibody as mentioned above and a suitable means of detection especially for detecting in food, food components, plant material or biological samples the presence of a gluten-derived peptide or epitope. Further part of the invention is an oligonucleotide primer set suitable for use in an amplification assay which is capable of binding to a nucleotide sequence coding for a gluten derived peptide or epitope or a DNA sequence which codes for a peptide which matches a consensus sequence according to the following formula (1), (2), (3), (4) or (5):
X₁-X₂-X₃-Q₄-X₅-P₆-Q₇-X₈-P₉-X₁₀ (1)
in which X₁₀ is F, Y, M, W, I or L and X₁, X₂, X₃, X₅, and X₈ are any amino acid;
X₁-X₂-X₃-Q₄-X₅-P₆-Q₇-X₈-X₉-X₁₀ (2)
in which X₉ is F, Y, M, W, I or L and X₁, X₂, X₃, X₅, X₈ and X₁₀ are any amino acid;
Q-X₁-P (3)
in which X₁is any amino acid;
Q-X₁-P-X₂ (4)
in which X₁ is any amino acid and X₂ is F,Y,W,M,L,I, or V;
Q-X₁-X₂-X₃ (5)
in which X₁ and X₂ are any amino acid and X₃ is F,Y,W,M,L,I or V.

Said primer set is preferentially used in a nucleotide amplification assay wherein food, food components, plant material or biological samples are tested for the presence of proteins or peptides which match any of the consensus sequences.

The hereinbefore mentioned detection methods may be used for the detection of cereals which are better suited to be used in food for celiac disease patients. It is also part of the invention to produce cereals which are better suited to be used in food for celiac disease patients by selecting cereals having a low amount of proteins or peptides matching the consensus sequences and crossbreeding them to produce cereals with even lower amounts.

Alternatively, the cereals can be produced by using genetic engineering techniques to diminish the natural amount of proteins or peptides matching the consensus sequences. Preferably the genetic engineering comprises transformation with recombinant DNA, most preferably homologous recombination or gene silencing, or induced mutation.

The preferred cereal is wheat.

Also part of the invention are cereal plants thus obtained and the use of such a cereal to prepare food for celiac disease patients.

Another part of the invention is a method to identify neo-epitopes from auto-antigens comprising comparing the sequence of an auto-antigens and searching for peptide sequences which match with the consensus sequences as defined before and subsequently screen biological material obtained from auto-immune disease patients for antibodies which bind to the peptide sequences or T cells which bind to the HLA-bound peptide sequences as identified in the previous step or peptides which are formed from said peptide sequences by deamidation of one or more Q residues.

Further, the invention comprises a method to inhibit the binding of a T cell receptor to an HLA-bound gluten-derived epitope or to an HLA-bound auto-antigen derived epitope comprising providing a blocking substance, preferably by blocking the binding of said epitopes to the HLA molecules.

Also comprised in the invention are analogues of the epitopes of the invention which are an antagonist for the activity of T cells recognising said-epitope.

A further part of the invention is a pharmaceutical composition comprising a peptide or an epitope according to the invention, which may preferably be used for the induction of tolerance or for the treatment of celiac disease or auto-immune disease.

A next part of the invention is a method to decrease sensitivity to auto-antigens by inhibiting the deamidation of said auto-antigen, preferably by inhibiting the function of tTG and most preferably at the site of presence of the auto-antigen.

Also part of the invention is the use of a protease inhibitor for preventing the generation of a peptide according to the invention or a polypeptide comprising such a peptide. For the similar purpose acid neutralising substances can be used. Further, the invention provides for a method to reduce the production of a auto-antigen derived peptide by inhibiting the expression of the auto-antigen from which it is derived.

### LEGEND TO THE FIGURES

FIG. 1. The influence of amino acid substitutions on deamidation of Q₂₀₈ and Q₂₁₆.

A. Simultaneous mass spectral analysis of four substitution analogs of the gliadin peptide before and after treatment with tTG as indicated. The untreated substitution analogs with a V, D, E, or F at position Q₂₀₈+3 have a mass of 1313, 1329, 1343 and 1361 Da respectively. After treatment with tTG two of those masses increase 1 Da, 1313 to 1814 and 1861 to 1362, indicating the conversion of a glutamine residue (128 Da) to a glutamic acid (129 Da) in the peptides with a V and F at position Q₂₀₈+3.

B. Overview of the influence of C-terminal ado acid substitutions on deamidation of Q₂₀₈ in the short version of the gliadin peptide and Q₂₁₆ in the long version of the peptide. The amino acids G₂₀₉, S₂₁₀, F₂₁₁, N₂₁₇, and Q₂₁₉ were substituted for all amino acids except lysine and cysteine and the effect on deamidation was determined. Substitution at other position in the peptide had only minor effects on deamidation of the Q₂₀₈ and Q₂₁₆-residues (not shown). (−) indicates no effect of the substitution; Substitutions that resulted in major differences in deamidation are designated with arrows: ↓↓ decrease of 70-100%, ↓ decrease of 30-69%.

^{↓} Substitution analogs were made in which the amino acids indicated were replaced by every natural amino acid except cysteine and lysine, because these residues could affect deamidation by formation of disulphide bridges and tTG driven cross-linking, respectively.

FIG. 2. Stimulation of gluten specific T cell lines by predicted peptides

A. A gluten specific T cell line isolated from a small intestinal biopsy of a celiac disease patient was tested against gluten, tTG-treated gluten and against the predicted peptides that were present in pools of five peptides each (pools 1-14). The odd numbers represent pools that contain the native peptides, whereas the even numbers represent corresponding pools that were treated with tTG. Strong reactivity of the T cell line was only observed with tTG-treated gluten and tTG-treated pools of peptides predicted by the XXXQXPQXPY algorithm.

B. Reactivity of the T cell line against the individual peptides from the T cell stimulatory pools. Bars indicated with an asterisk are considered positive (Stimulation Index>3).

FIG. 3

tTG inhibitor

FIG. 4

Inhibition of tTG induced deamidation of a gluten peptide by the inhibitor depicted in FIG. 3.

### DETAILED DESCRIPTION

In celiac disease both T-cells reactive with (epitopes derived from) gluten and antibodies reactive with the enzyme tTG (tissue transglutaminase) have been found. It is hypothesized that the epitopes from gluten are the causative factor of the disease. The antibodies specific for the endogenous tTG can be explained because the enzyme crosslinks with gluten proteins or parts of those, which complex then will be recognized by a B-cell expressing tTG specific antibodies on its cell surface. The complex, including the gluten will then be processed by the B-cell to present the antigenic determinants to gluten specific T-cells, thereby delivering specific T-cell help, resulting in the secretion of tTG-specific antibodies.

We have now determined the patterns of deamidation in gluten, and find that this is highly dependent on the spacing between the glutamine and proline residues. This knowledge can be used to predict novel T cell stimulatory gluten peptides. Moreover, our results provide an explanation for the toxicity of gluten and the related hordeins in barley and the secalins in rye, while other food proteins, in particular the avenins in oats, are not toxic for CD patients. Also, the finding can explain the formation of neo-epitopes in autoimmune diseases such as RA (rheumatoid arthritis), MS (multiple sclerosis), SLE (systemic lupus erythomatosus), SS (Sjogren syndrome) and DB (diabetes),

It has been known in the prior art that the tTG enzyme affects the deamidation of a glutamine (Gln; IUPAC code: Q) moiety on the target peptide into a glutamic acid moiety (Glu; IUPAC code: E). Gluten proteins, such as gliadin, glutelin, hordein and secalin, however, contain very many Q residues (in gliadin approximately 30-40% of all amino acid residues) and it has been shown that not all Q residues are susceptible to deamidation by tTG in the same manner. Thus, it is clear that the near environment of the Q residue on the target protein (be it in the primary, secondary or tertiary structure of the protein) influences the availability of the Q residue to act as a substrate for the enzyme. It has been shown earlier (van de Wal, 1998a) that in one of the gliadin peptide parts which is able to stimulate T-cells (PSGQ₂₀₈GSFQPSQQ₂₁₆NPQA) the Q residues at position 208 and 216 are deamidated, while the Q residues at positions 212, 215 and 219 are less or not amidated. From detailed studies as are shown in the experimental part it has now been established that the presence of proline (Pro, IUPAC code P) in the neighbourhood of the Q residue is a key determining factor of the susceptibility of said residue to deamidation by tTG. In the sequences QP and QXXP (X is any amino acid) no or very little deamidation of the Q-residue was observed In contrast, in the sequences QXP, QXXF(Y, W, L, I, M or V) and QXPF(Y,W, L, I, M, or V) efficient deamidation of the Q-residue was observed. Also, a minimum of three amino acids C-terminal of the target Q-residue is required for deamidation. Based on this finding two consensus sequences have been developed which have a very high predictive value for susceptibility to tTG catalysed deamidation.

On the basis of these results two search algorithms were defined, specifically aimed to aid in the search for HLA-DQ2-binding peptides in the gluten databases that would be a potential target for gluten specific T cells isolated from celiac disease patients. In these algorithms the deamidation of Q redidues at position p7 and/p4 is predicted to mediate binding to HLA-DQ2. The first algorithm predicts deamidation at p4 and p7 and reads as follows:
X₁-X₂-X₃-Q₄-X₅-P₆-Q₇-X₈-P₉-X₁₀ (1)
in which X₁₀ is F, Y, M, W, I or L and X₁, X₂, X₃, X₅, and X₈ are any amino acid.

The second algorithm predicts deamidation at p4 and reads as follows:
X₁-X₂-X₃-Q₄-X₅-P₆-Q₇-X₈-X₉-X₁₀ (2)
in which X₉ is F, Y, M, W, I or L and X₁, X₂, X₃, X₅, X₈ and X₁₀ are any amino acid.

On basis of these algorithms (also called herein consensus sequences) novel peptide parts of gluten proteins have been discovered, which are able to act as substrate for tTG and which, upon deamidation, have antigenic activity as measured by their ability to stimulate gluten specific T cell lines. From hereon these novel peptides will be called gluten-derived peptides. These peptides have the amino acid sequences: QQPYPQQPQQPFPQ, QQPFPQQPQQPFPQ, PFPQTQQPQQPFPQ, PFPQLQQPQQPFPQ, QFPQTQQPQQPFPQ, QLPFPQQPQQPFPQ, PFPQPQQPQQPFPQ and PFPQSQQPQQPFPQ.

It should be clear that also C-terminally and N-terminally extended peptides comprising the herebefore mentioned sequences are part of the invention. Thus, the invention comprises amino acid sequences of 14-40 amino acids which comprise the above mentioned sequences.

Also part of the invention are the epitopes derived from these gluten-derived peptides by deamidation of one or more Q residues. These gluten-derived epitopes thus have the amino acid sequences: QQPYPEQPQQPFPQ, QQPFPEQPQQPFPQ, PFPQTEQPQQPFPQ, PFPQLEQPQQPFPQ, QFPQTEQPQQPFPQ, QLPFPEQPQQPFPQ, PFPQPEQPQQPFPQ, PFPQSEQPQQPFPQ, QQPYPEQPEQPFPQ, QQPFPEQPEQPFPQ, PFPQTEQPEQPFPQ, PFPQLEQPEQPFPQ, QFPQTEQPEQPFPQ, QLPFPEQPEQPFPQ, PFPQPEQPEQPFPQ, PFPQSEQPEQPFPQ, QQPYPQQPEQPFPQ, QQPFPQQPEQPFPQ, PFPQTQQPEQPFPQ, PFPQLQQPBQPFPQ, QFPQTQQPEQPFPQ, QLPFPQQPEQPFPQ, PFPQPQQPEQPFPQ and PFPQSQQPEQPFPQ, wherein further Q residues may be deaminated into an E residue.

Furthermore, it is known from the prior art that neo-epitopes derived from presumed antigenic auto-immunogens can be a mechanism to explain the occurence of major auto-immune diseases such as RA (rheumatoid arthritis), MS (multiple sclerosis), SLE (systemic lupus erythomatosus), SS (Sjogren syndrome) and DB (diabetes). Thus far, it has not been recognised which factor is responsible for the existence of these neo-epitopes. Surprisingly now, when the amino acid sequences of the antigens which are deemed to play a role in the above mentioned auto-immune diseases are screened with the following consensus sequence(s): QX₁P, QX₁PX₂ or QX₁X₁X₂ (wherein X₁ is any amino acid and X₂ is F, Y, W, M, L, I or V) or the sequences as described above in (1) or (2) in all antigens listed in Table 5 peptide sequences are found which are prone to deamidation by tTG. It is now postulated that in these diseases tTG is able to recognise the antigens or processed antigens and that the peptide part which is recognised has been deamidated and will function as a neo-epitope which will evoke a T-ell response.

Table 5 shows the antigens and the amino acid sequences comprised in these antigens which are thought to be involved in the above mentioned auto-immune diseases. Thus, a further aspect of the invention comprises the auto-antigen derived peptides listed in Table 5, including the peptides in which the Q residue is replaced by an E at position 10 (the central Q residue).

To a person skilled in the art it is known that peptides of variable length can bind to HLA-molecules. The relevant peptides can therefore be longer or shorter versions as those shown and might even extend to N-terminally and/or C-terminally extended sequences that are not shown but present in the proteins from which these peptide sequences are derived. Thus, the invention comprises amino acid sequences of 8 amino acid residues (8-mers) derivable from and comprising the central Q residue of those sequences as listed in Table 5. For example then (taking the first sequence of Table 5: SRFSWGAAEGQRPGFGYGGR) the peptides of the invention would include the following 8-mers: FSWGAEGQ, SWGAEGQ, WGABGQRP, GAEGQRPG, AEGQRPGF, FGQRPQFG, GQRPGFGY and QRPGFGYG. Further comprised in the invention are all peptides of 40 amino acid residues or less than 40, which comprise one or more of the above mentioned 8-mers. For ease of reference these peptides further are denominated as auto-antigen derived peptides.

Analogous to the situation with the gluten-derived peptides, also here in case of the auto-antigen derived peptides, neo-epitopes can be defined in which the central Q residue in the peptides listed in Table 5 has been deamidated into an E residue.

Since these consensus sequences now make it possible for the first time to predict amino acid sequences which upon deamidation by tTG act as epitopes in celiac disease or other auto-immune diseases, the information can also be used in various assays and therapies relating to these diseases.

The most promising application in the area of celiac disease is to screen foodstuffs for the presence of sequences matching with the consensus sequence(s).

A first embodiment for such a screening approach which is included in the invention is the use of (HLA-DQ2 and HLA-DQ8) restricted T cells which recognise the gluten-derived epitopes, or the receptor of such T cells. Such T cells are obtainable by methods well known to those &killed in the art. A preferred way to obtain these T cell is to collect T cell biopsies from celiac disease patients, which show typical clinical symptoms and/or are responsive in an anti-endomysium test. These biopsies then are cultured with either a (chymo)trypsin/pepsin digest of gluten optionally additionally treated with tTG. Cultures that show evidence of T cell proliferation are expanded and tested for specificity to the gluten-derived epitopes of the invention. From these gluten specific T cells optionally clones can be generated and the T cell receptor of such T cell clone can be cloned and transfected or transduced into other appropriate cells or cell lines to yield a functional T cell receptor. These T cells or T cell receptors can be used to screen foodstuff for the presence of epitopes. They can also be used to screen plant material, especially varieties of cereals such as wheat, to check the occurrence of said epitopes.

Preferably in these types of assays the protein material in the food or in the plant varieties is treated with a protease such as trypsin, chymotrypsin or pepsin and further treated with the enzyme tTG to mimic the protein degradation in the human body.

Preferably such a screening is performed with a panel of T cells having specific reactivity to different epitopes. In this way the safety of food for celiac disease patients can be tested reliably. Also in this way it is possible to detect varieties of cereals which would be suitable for incorporation into food products for celiac disease patients.

Next to the use of specific T cells another embodiment comprised in the invention for assaying the safety of foodstuffs or the suitability of cereal varieties for food processing is to use antibodies which are capable to bind the gluten-derived peptides or gluten-derived epitopes of the invention. Such antibodies are for instance obtainable by immunising an immunocompetent animal with the gluten-derived peptides or gluten-derived epitopes according to the invention or an immunogenic fragment thereof, coupled to a suitable carrier protein if necessary, and harvesting polyclonal antibodies from said animal, or obtainable by other methods known in the art such as by producing monoclonal antibodies, or (single chain) antibodies or binding proteins expressed from recombinant nucleic acids derived from a nucleic acid library, for example obtainable though phage display techniques.

With such an antibody, the invention also provides for an immunoassay comprising an antibody according to the invention. Various types of immunoassays are available within the art, for example ELISA (Enzyme Linked Immuno Sorbent Assay) or Western blotting.

Analogous to assays with specific T cells as discussed above, also in the case of antibodies preferably a panel of antibodies is used.

A further example for assaying the safety of food and to identify cereal varieties that lack the predicted T-cell stimulatory sequences is the use of nucleotide primer based amplification technology. In such a method oligonucleotide primers are developed corresponding with DNA coding for the specific gluten-derived peptides or the general amino acid consensus sequence(s). These primers then are brought into contact with DNA or RNA isolated from the food or the plants and the mixture is subjected to a treatment to amplify the nucleotide sequences to which the primers bind. These DNA or RNA amplification techniques, such as PCR and NASBA, are known and readily available to a person skilled in the art. The amplification step will result in a specific multiplication of the nucleotide fragment to which the primers bind and, in the present case, thus is indicative of the occurrence of even a single copy of the nucleotide coding for a gluten-derived peptide or a peptide which matches the consensus sequence.

It is acknowledged that to develop an assay testing for the safety of food for celiac disease patients more than one T cell clone, more than one set of primers or more than one antibody is needed. In yet another embodiment the invention thus comprises assays having an array of antibodies as discussed above, all recognising different sequences, which fall within the consensus sequence(s) of the invention. Similarly, the invention also provides for a multiple of oligonucleotide primer sets which are able to bind with different nucleic acids coding for amino acid sequences which fall within the consensus sequence(s) of the invention.

In yet another embodiment the invention provides a diagnostic kit comprising an antibody according to the invention or a set of oligonucleotide primers according to the invention and a suitable means of detection. Such a diagnostic kit is, for example, very useful for detecting in food, food components or samples from (suspected) patients the presence of a gluten-derived peptide according to the invention or a peptide sequence involved in food-related immune enteropathy (for example: celiac sprue, tropical sprue, giardiasis or food allergies of childhood) matching the consensus sequence(s) of the invention. At present such a quantitative and qualitative diagnostic kit determining the presence and/or amount of gluten-derived peptide is not available. Currently two different assays are used for gluten detection. One assay determines the nitrogen content of food (components) as a measure for the presence of gluten. The other assay utilises gluten specific antibodies in ELISA systems. However, both assay systems do not test for the toxic gluten-derived peptides involved in food-related immune enteropathy. A diagnostic kit comprises, for example, an antibody according to the invention specifically recognising a toxic gluten-derived peptide involved in food-related immune enteropathy. Another advantage of the diagnostic kit as described in the present application is the capability of testing food (components) which cannot be tested or cannot be tested reliably by the currently used gluten assays. The existing assays are hardly informative when food (components) contain significant amounts of other nitrogen containing compounds (e.g. other proteins) or when food (components) contain partially hydrolysed gluten proteins that are not recognised by antibodies currently used in ELISA-kits. Examples of food (components) or which the existing assays are troublesome are beer, melassis and soy sauce. In addition, the existing assays lack the level of sensitivity required for many applications.

Preferably a diagnostic kit according to the invention uses different kinds of T cells, T cell receptors, antibodies or oligonucleotide primer sets according to the invention or different antibodies, each capable of recognizing another gluten-derived peptide involved in food-related immune enteropathy. Thereby multiple gluten-derived peptides involved in food-related immune enteropathy are detected. In the art different kinds of means of detection are available and the skilled person knows how to select a proper means of detection. Examples are chromogenic or fluorigenic substances. The invention thus provides methods and means for the monitoring of a T-cell reactive component in food, food component or samples from (suspected) patients.

Another aspect of the invention is the use of the consensus sequence(s) of the invention to search for peptide sequences which could invoke immune responses. One of the possible ways to achieve this is to screen existing protein or nucleotide databases and to identify amino acid sequences or nucleotide sequences coding for amino acid sequences which match with the consensus sequence of the invention. Methods for identification of matching sequences in databases, such as the BLASTN computer program (which is publicly available, or instance through the National Centre for Biotechnological Information, accessible via the internet on http://www.ncbi.nlm.nih.gov/) are well known to persons skilled in the art.

Furthermore the invention provides a method to select and/or breed a cereal which has a decreased amount of or is substantially free of peptide sequences which are toxic for celiac disease patients. Cereals are selected for the absence of peptide sequences matching the consensus sequence(s) of the invention according to any of the above described methods. Such selected cereals are than included in breeding schemes using normal agricultural breeding methods to develop cereal varieties useful or food production which exhibit a better tolerance to celiac disease patients. Cereal, in this application, relates to gram or related grasses or plants that produce it and to the (prepared) foodstuff. In particular wheat gluten, but also rye, and to a lesser extent barley and oat are preferred cereals.

Because the consensus sequence(s) for determining peptides which cause immunological effects are disclosed herein, one is now also able to genetically modify the genome of cereals to generate new cereals with a decreased source of toxic peptides. Modifications are, for example, generated by point-mutations in the nucleic acid sequence coding for the gluten proteins or are generated by replacing, e.g. through homologous recombination, a sequence coding for an amino acid sequence which matches the consensus sequence(s) of the invention by another sequence not giving rise to amino acid sequences matching the consensus sequence(s). Further, expression of a protein which contains toxic peptide sequences may be prohibited by gene silencing. Numerous methods for gene silencing in plants, such as antisense expression, sense co-suppression and RNAi are known in the art and readily applicable by a person skilled in the art.

Since, according to this invention, the presence of proline residues determine for a large part the susceptibility of a peptide for deamidation by tTG, it is preferred to change the nucleotide sequences coding for the amino acid sequences in such a way that the proline residues of peptide fragments which match the consensus sequence are changed into other residues. Such conservative substitutions of the proline residues are possible without substantially affecting the chemical and physical properties that are so desired in gluten. Cereal plants, thus genetically modified, can again be used in cross-breeding programmes in order to come to a variety which can be used in agriculture.

A cereal selected and/or bred according to a method of the invention is used to prepare food low or preferably free of toxic peptides involved in food-related immune enteropathy.

Further, the invention also comprises methods to identify possible toxic peptides from auto-antigens. According to any of the above-mentioned methods peptide sequences from antigens which are deemed to play a role in auto-immune diseases can be detected. Table 5 lists a number of predicted toxic peptide sequences comprised in proteins which are believed to play a role in diseases such as diabetes, rheumatoid arthritis, multiple sclerosis, systemic lupus erythomatosus, Sjogren syndrome and the like. It is now possible, using the information from the consensus sequence(s) of the invention to identify toxic peptides from yet other auto-antigens. Neo-epitopes can be formed from these peptide sequences by deamidation with tTG. It is now possible for a person skilled in the art to investigate whether these neo-epitopes indeed play a role in auto-immune diseases. A simple way to do this is to screen serum, lymph or any other biological material from auto-immune disease patients for the presence of T calls and/or antibodies against such neo-epitopes (which do not, or to a lesser extent, occur in non-autoimmune diseased control patients). This would further allow for a diagnostic method for assessing disease or predisposition to disease on basis of the occurrence of said antibodies.

Furthermore the invention provides a method to decrease or more preferably completely inhibit the binding of a T-cell receptor to a gluten-derived epitope or neo-epitope involved in auto-immune disease comprising providing a blocking substance of said Tell receptor. By decreasing and more preferably completely inhibiting the binding of the T-cell receptor to an HLA-bound gluten-derived epitope or neo-epitope, effects of the immune disease are decreased or preferably completely diminished. Such a blocking substance prevents the association of the T-cell receptor with the HLA-bound gluten peptide and prevents the T-cell receptor in its activity. For example such a blocking substance is a natural or synthetic variant of a gluten-derived epitope or neo-epitope according to the invention, for example an, altered peptide ligand, and is especially well suited for use in a therapy against gluten-derived peptide or auto-antigen derived peptide sensitivity. It is clear that the binding of said blocking subs to a T-cell receptor does not allow functional signalling of a T-cell comprising said T-cell receptor.

Furthermore gluten-derived peptides or auto-antigen derived peptides according to the invention are used to prepare therapeutic agents capable of eliminating a subset of cells, directly or indirectly, especially gluten-sensitive or auto-antigen sensitive T-cells. This means that an agent, which typically comprises a gluten-derived peptide or an auto-antigen derived peptide according to the invention as recognised selectively by T-cells, which agent induces elimination of the cells recognising said peptide, is administered to the patient. Such an agent-most typically also comprises a toxic moiety to mediate the elimination of the specific T cells.

In yet another embodiment the invention provides a method to decrease (or more preferably completely inhibit) the binding of gluten-derived peptides involved in food-related immune enteropathy (for example celiac sprue, tropical sprue, giardiasis or food allergies of childhood) or auto-antigen derived peptides involved in auto-immune diseases to T cell receptor molecules comprising providing substances that block the binding of said peptides to said receptor molecules. By decreasing and more preferably completely inhibiting the binding of a gluten-derived epitope to an HLA-DQ molecule or the binding of a neo-epitope to its receptor at the T cell, effects of the immune related disease are decreased or more preferably, completely diminished.

A blocking substance associates with, for example, a T cell receptor molecule and prevents the epitope to associate with said molecule. Such a blocking substance is, for example, a natural or synthetic variant of a gluten-derived epitope or neo-epitope. It is clear that the binding of said blocking substance to said T cell receptor is such that it decreases or more preferably completely diminishes the binding of a gluten-derived epitope or neo-epitope to said receptor molecule. Another way to decrease or more preferably to completely inhibit the binding of a gluten-derived epitope to an HLA-DQ molecule or a neo-epitope to its T cell receptor, is by providing an antibody which associates with said gluten-derived epitope or neo-epitope.

In yet another embodiment the invention provides a method to detect and/or enumerate T-cells bearing a T-cell receptor according to the invention comprising tetrameric complexes of HLA molecules and a gluten-derived epitope or neo-epitope according to the invention. Methods to arrive at such a tetrameric complex are known in the art. In another embodiment the invention provides a method to detect and/or enumerate T-cells bearing a T-cell receptor according to the invention comprising (synthetic) liposomes comprising complexes of HLA molecules and a gluten-derived epitope or neo-epitope according to the invention. Methods to arrive at such complexes are known by the person skilled in the art. These data provide a novel tool for detection and enumeration of T-cells comprising a T-cell receptor according to the invention.

In another embodiment the invention provides a pharmaceutical composition comprising a gluten-derived peptide or auto-antigen derived peptide according to the present invention. Such a pharmaceutical composition is used for the induction of tolerance against said gluten-derived or auto-antigen derived peptide. For tolerance induction doses of this peptide according to the invention are given repeatedly, for instance intravenously, but other routes of administration are suitable too. Another possibility is the repeated oral or nasal administration of such a peptide. Such a peptide according to the present invention is given alone, or in combination with other (toxic) gluten-derived peptides and/or auto-antigen derived peptides, or as part of larger molecules, or coupled to carrier materials/molecules. A pharmaceutical composition comprising a peptide according to the present invention can also be used for elimination of a certain subset of T-cells or for the treatment of celiac disease or auto-immune diseases. Preferably such a pharmaceutical composition according to the present invention contains various, different kinds of, gluten-derived or auto-antigen derived peptides.

In yet another embodiment use is made of a protease inibitor or of acid neutralizing substances for preventing the generation of a gluten-derived or auto-antigen derived peptide according to the invention or a polypeptide comprising such a peptide. The proteins which comprise the gluten-derived or the auto-antigen derived peptides are not capable of binding to an HLA molecule directly and must first be processed by proteases to provide a peptide or peptides capable of binding to an HLA molecule. Gluten-derived peptides and polypeptides comprising a gluten-derived peptide according to the invention are bound to HLA-DQ molecules and are thereby recognized by a T-cell receptor. By preventing the formation of gluten-derived peptides, binding to HLA-DQ molecules and recognition by T-cell receptors is prevented. One way to prevent a gluten-derived peptide from being generated is by inhibiting the enzyme (for example by protease inhibitors) which is capable of processing the proteins from which the gluten-derived peptides are derived (for example glutenins and/or gliadins). Another way to prevent the gluten-derived peptides from being generated is inactivating the enzyme, which is capable of processing the proteins from which the gluten-derived peptides an derived by providing neutralizing substances. Pepsin and trypsin are examples of enzymes that work under acidic conditions and by providing neutralizing substances the effects of these enzymes are diminished or more preferably completely inhibited.

In the case of auto-antigen derived peptide or polypeptides comprising those, inhibition of the reaction which deamidates said peptides to form neo-epitopes would yield a decrease of the antigenic effect of the auto-antigen. Preferably said inhibition is accomplished by local inhibition of the enzyme tTG at the site(s) where normally these neo-epitopes are formed. For celiac disease, site specific administration in the gut would be the method of choice. In RA, the specific site to inhibit the formation of neo-epitopes would be the joints, while for Sjogren syndrome the site would be the tear and salivary glands. Inhibition of the enzyme can be accomplished by specific inhibitory compounds, e.g. by antibodies binding to the enzyme in such a way that the enzymatic function is decreased. Another approach is to design specific compounds on the basis of the defined specificity of tTG for particular gluten sequences like QLPF and QXXF. Site-specific action of such compounds can be achieved by local application of the inhibitor. e.g. by injection. An example of a tTG inhibitor/blocker is disclosed herein within the experimental part. A lead compound was designed on the basis of an octapeptide, sequence QPQLPYPQ, known to be an tTG substrate {Arentz-Hansen, H., R. Korner, O. Molberg, H. Quarsten, W. Vader, Y. M. Kooy, K. E. Lundin, F. Koning, P. Roepstorff, L. M. Sollid, and S. N. McAdam. 2000. The intestinal T cell response to alpha-gliadin in adult celiac disease is focused on a single deamidated glutamine targeted by tissue transglutaminase. J. Exp. Med. 191:603-612} and containing the sequence QLPY which allows the transformation of the indicated glutamine (Q) into glutamate. On basis of the structure as disclosed in FIG. 3, a person skilled in the art is capable of introducing modifications which leave the inhibitor function intact but which would make the compound more suitable for therapeutic applications. For example, an optimum between pharmacological efficacy and ease of synthesis will be established by trimming the oligopeptide sequence since previous results indicate that the minimal peptide fragment required for recognition by tTG is a peptide from 3 to 4 amino acids in length {as disclosed herein and in Vader, L. W., A. De Ru, W. Y. van der, Y. M. Kooy, W. Benckhuijsen, M. L. Mearin, J. W. Drijfhout, P. Van Veelen, and F. Koning. 2002. Specificity of tissue transglutaminase explains cereal toxicity in celiac disease. J. Exp. Med. 195:643-649}. Furthermore, recognition of the inhibitor by tTG may be enhanced by modifying the amino acid composition and sequence. First of all, amino acid sequences having the sequence QXP, QXXF or QXPF, as discussed earlier, are especially useful, since these sequences are shown to be target sequences for the enzyme. Further, the binding strength of the inhibitor can be modulated by incorporating different electrophilic traps, either reversible (aldehyde, boronate) or irreversible (chloromethyl ketone, vinylsulfone, epoxyketone). Protease resistance is another important point which can be addressed by replacement of several selected peptide bonds with isosteres (e.g. N-alkylamide, sulfonamide, urea or alkene moieties). In this respect, it is of interest to study the effect of incorporation of sugar amino acids in the peptide sequence.

In another embodiment, the invention provides a tTG inhibitor/blocker characterised in that said inhibitor/blocker is derived from a peptide sequence as disclosed herein and further comprises an electrophilic trap. In yet another embodiment, the invention provides a tTG inhibitor/blocker characterised in that said inhibitor/blocker is derived from the peptide QPQLPYPQ and further comprises an electrophilic trap. Preferably, the electrophilic trap replaces the carboxamide moiety of a glutamine that is converted into a glutamate by the action of tTG. As disclosed herein within the experimental part, replacement of the carboxamide moiety of the glutamine at position 3 in the peptide QPQLPYPQ by a vinylogous ester as an electrophilic trap leads to effective inhibition of tTG. It is clear to a person skilled in the art that other electrophilic traps can also be used. An example of a tTG inhibitor/blocker is depicted in FIG. 3.

The invention also provides a pharmaceutical composition comprising a tTG inhibitor/blocker as described above or a functional equivalent and/or functional fragment thereof. Furthermore, the invention also provides use of a tTG inhibitor/blocker as described above or a functional equivalent and/or functional fragment thereof or the preparation of a medicament for the treatment of celiac disease or auto-immune disease.

The invention will be explained in more detail in the following detailed description which is not limiting the invention.

Materials and Methods

Peptides and tTG treatment. Peptides were synthesised by standard Fmoc chemistry on a multiple peptide synthesizer (SyroII, MultiSynTech GmbH, Witten, Germany). Integrity of synthetic peptides was checked by rpHPLC and mass spectrometry. Tissue transglutaminase (tTG) treatment was performed by incubating the peptides (500 μg/ml) with the enzyme (100 μg/ml, Sigma, Zwijndrecht, NL; T-5398) at 37° C. for 4 h minimum, in 50 mM triethylamine-acetate pH 6.5, 2 mM CaCl₂.

Mass spectrometry. Electrospray ionisation mass spectrometry was performed on the synthetic gluten peptides before and after tTG treatment, using a Q-TOF hybrid mass spectrometer (Micromass, Manchester, UK). Precursor ions were selected with the quadrupole window set to 3 Da and fragments were collected with high efficiency with the orthogonal time of flight mass spectrometer. The collision gas applied was argon (pressure 4×10⁻⁵ mbar) and the collision voltage approximately 30 V. Deamidation of glutamine residues in synthetic gluten peptides was determined by mass spectrometric analysis as described previously (van de Wal, 1998a). Deamidation of a glutamine residue results in an increase of 1 Da. These conversions were assigned to particular glutamine residues by comparison of the fragmentation spectra of tTG treated and non-treated peptides.

Database searching. The program PeptideSearch was used for pattern searches Mann, M. and Wilm, M., Anal. Chem. 66: 4390-4399, 1994). The patterns were composed on the basis of the newly identified specificity of tissue transglutaminase in combination with the previously identified HLA-DQ2 peptide binding motif (Johansen 1996; van de Wal, 1996). For database searching a selected subset of wheat proteins from a compilation of the Swiss Prot databank, SPTREMBL and PIR was used. This compilation contained 982 proteins.

T cell clones T cell lines. Gluten specific T cell lines were generated from small intestinal biopsies of celiac disease patients as described before (van de Wal, 1998a).

T cell proliferation assays. Proliferation assays were performed in duplicate in 150 μl RPMI1640 (Gibco, Breda, NL) supplemented with 10% human serum in 96-well flat-bottomed plates (Falcon) using 10⁴ T cells stimulated with 10⁵ irradiated PBMCs (3000 RAD) in the presence or absence of antigen (1-10 μg/ml). After 48 hours at 37° C., cultures were pulsed with 0.5 μCi of ³H-thymidine and harvested 18 hours thereafter as described previously (van de Wal, 1998b).

Results

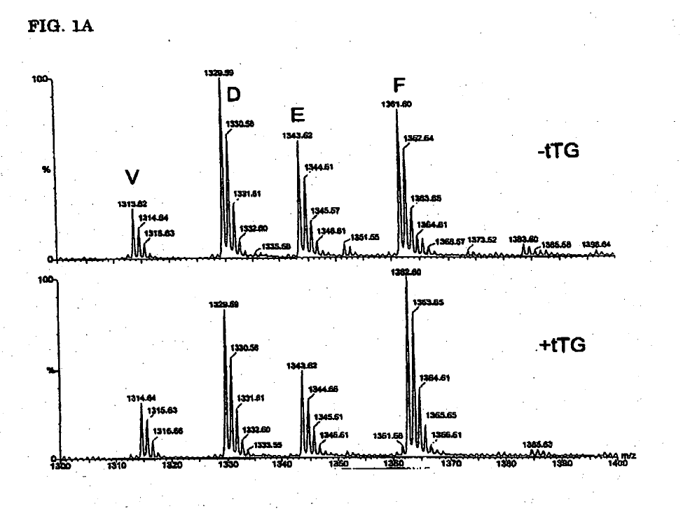

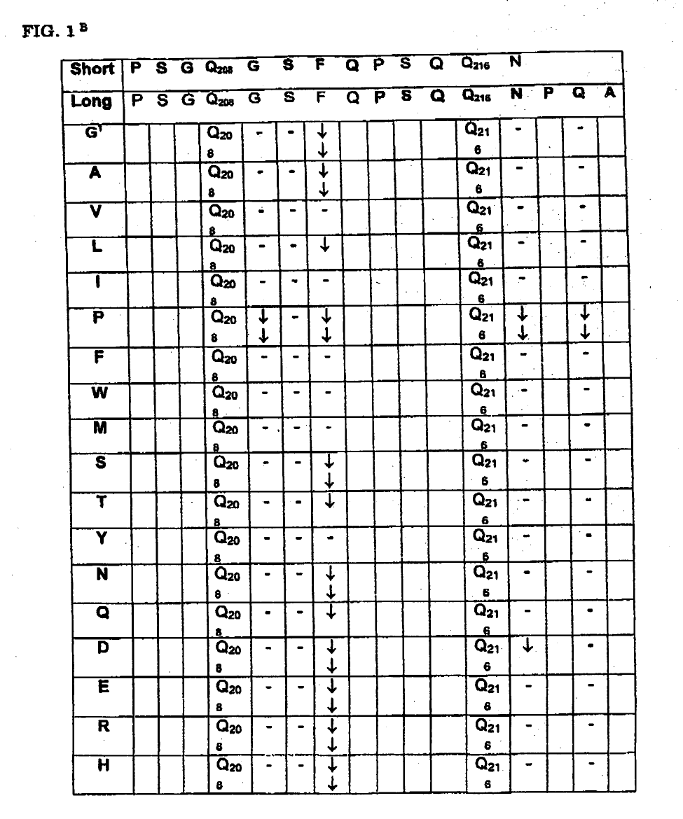

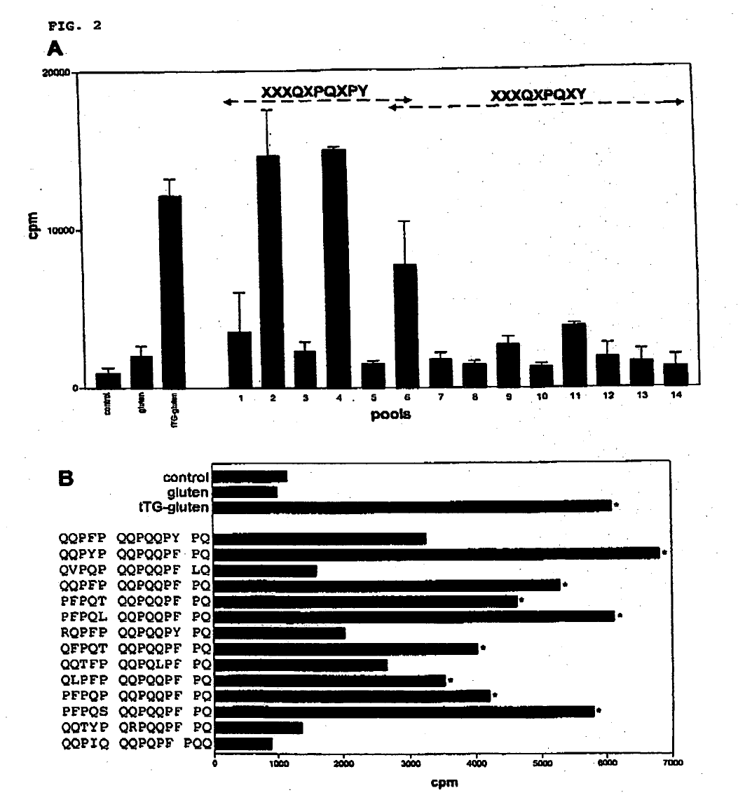

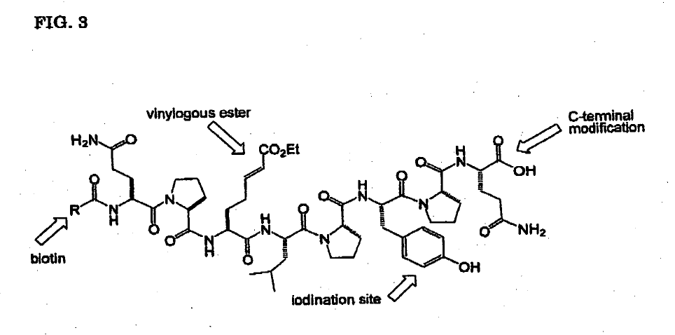

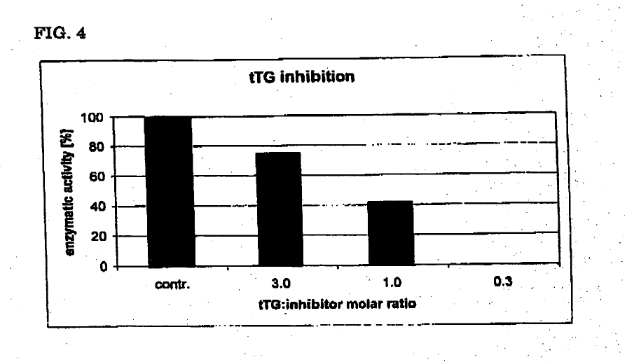

The specificity of tTG. Previously we have characterized an HLA-DQ8 restricted, T cell stimulatory gliadin peptide (van de wal, 1998a;1998b). In the core of this gliadin peptide (sequence PSGQ₂₀₈GSFQPSQQ₂₁₆NPQA) the Q₂₀₈ and Q₂₁₆ are deamidated by tTG, whereas positions Q₂₁₂, Q₂₁₅, and Q₂₁₉ are not (van de Wal, 1998a). Preliminary studies indicated that the nature of C-terminal flanking amino acids influenced deamidation of glutamine residues (not shown). We have now investigated this systematically by analyzing the deamidation in amino acid substitution analogs of a shorter version of the gliadin peptide (sequence: PSGQ₂₀₈GSFQPSQQ₂₁₆N). In this peptide only the Q₂₀₈ is deamidated by tTG treatment (FIG. 1). An example of this type of analysis shows the simultaneous mass spectrometric analysis of 4 of these substitution analogs, that have either the amino acid V, D, E, or F at position Q₂₀₈+3, and reveals the distinct masses corresponding to these peptides (FIG. 1a). Upon treatment of the peptides with tTG two of the masses increase by 1 Da, indicating conversion of a glutamine into a glutamic acid (FIG. 1a). Subsequent analysis of collision induced fragments of hem individual peptides (not shown) was used to verify that deamidation occurred at Q₂₀₈ in these peptides. These results demonstrate that in the substitution analogs with the amino acids V and F at position Q₂₀₈+3, complete deamidation of Q₂₀₈ is found while in the peptides that carry a D or E at Q₂₀₈+3, no deamidation of Q₂₀₈ takes place. The complete analysis demonstrates that at only two positions in the peptide substitutions had effect on deamidation of Q₂₀₈ (not shown and FIG. 1b) Fist, the replacement of Q₂₀₉ by a P abolished deamidation of Q₂₀₈. Second, several amino acid substitutions at F₂₁₁ had a strong negative effect on deamidation of Q₂₀₈, in particular replacements of F₂₁₁ for amino acids with significantly smaller or charged side chains or replacement by proline (FIG. 1a, b).

Next, we carried out a similar analysis to determine the influence of amino acid substitutions on the deamidation of Q₂₁₆ in a longer version of the peptide (FIG. 1b). The results demonstrate that the substitution of N₂₁₇ with a P abolishes deamidation of Q₂₁₆, a finding that underscores the observation that in a QP sequence the Q is not a target for tTG. Moreover, the substitution of Q₂₁₀ with a P had a negative effect on deamidation of Q₂₁₆, confirming the negative influence of the P in the sequence QXXP (X stands for any amino acid). Amino acid substitutions (with the exception of the P substitution) at Q₂₁₆+3, however, had no effect on deamidation of Q₂₁₆, a result that is in contrast to the strong influence of amino acid substitutions at position Q₂₀₈+3 on deamidation of Q₂₀₈. A marked difference between the sequence Q₂₀₈GSF and Q₂₁₆NPQ is the presence of a P in the latter sequence at Q+2 suggesting that in the sequence QXP the Q is a good target for deamidation and the nature of the amino acid at Q+3 is much less important (see also below). Together the results indicate that in the sequences QP and QXXP the Q is not a target for tTG. In contrast the sequences QXP, QXX(F, Y, W, M, L, I, or V) and QXP(F, Y, W, M, L, I, or V) favor deamidation of the Q (Table 1). To verify these “rules” for deamidation by tTG, a large panel of gluten peptides was synthesized, treated with tTG and analyzed by mass spectrometry (Table 2). Without exception we fund that a Q preceding a P is not deamidated by tTG while in the majority of cases a P at Q+3 also inhibited deamidation. In contrast, a bully/hydrophobic residue at Q+3, and P at Q+2 allowed good deamidation. In the case that two opposite rules coincide, as is found in peptide 20 (LQQPQQ₆PQFQPQQQF) the negative effect of QP↓ overrules the positive effect of QXXF↑ at position 6. Together, these results confirm the defined patterns. Proline thus plays a crucial role in the deamidation of gluten peptides.

Prediction of novel gluten epitopes The peptide binding motifs of the disease associated molecules HLA-DQ2 and HLA-DQ8 display preferential binding of negative charges at anchor positions (Johansen, 1996; van de Wal, 1996, Kwok, 1996). HLA-DQ2 has a strong preference for a negative charge at p4 and p7. Strikingly, at p6 a proline is one of the preferred amino acids in the HLA-DQ2 peptide binding motif that could thus target specific deamidation at p4 (Table 3). Therefore we combined the HLA-DQ2 peptide binding motif with the specificity of tTG to construct an algorithm that could be used to predict novel T cell stimulatory gluten peptides from the gluten database (Table 3). In this algorithm glutamine residues were introduced at the putative DQ2 binding positions p4 and p7 that serve as potential target sites for tTG to allow introduction of a negative charge at these positions. Next, proline was selected for p6 to promote deamidation at Q₄, which is also consistent with the requirements for peptide binding at this position (Table 3). For the p9 position two options were designed. In the first option bulky/hydrophobic residues were selected for p9, according to the peptide binding motif. In the second option, a proline residue was placed at p9, followed by bulky/hydrophobic at p10. This option does not agree with the peptide binding motif but should allow optimal deamidation at the Q₇ according to our QXPF rule. The remaining positions p1, p2, p3, p5, and p8 were not defined (Table 3). These two algorithms were used to search in the gluten database. A total of 98 matches were obtained, which corresponded to 27 unique gluten sequences. These 27 unique gluten peptides were synthesized, and deamidated by tTG. Mass spectral analysis confirmed deamidation of the predicted Q₄- and Q₇-residues (not shown).

The native and deamidated peptides were pooled in pools of five peptides each, and tested in T cell proliferation assays with three gluten specific T cell lines isolated from small intestinal biopsies of celiac disease patients. Two of these T cell lines responded to stimulation by several of the deamidated peptide pools (FIG. 2a, results shown or one of the T cell lines). The responses were exclusively found against pools that contained peptides predicted by the second algorithm, X X X Q₄ X P₆ Q₇ X P₉ (Y,F,W,I,L)₁₀ (FIG. 2a, b). Subsequently, the T cell line was tested against the individual peptides of the positive pools, and found to respond to 8 out of 13 peptides tested (FIG. 2b).

In addition we performed searches with a less strict variant of algorithm 2, X X X X₄ X P₆ Q₇ X P₉ (Y,F,W,I,L)₁₀ which predicts the deamidation of only one Q-residue. This search yielded 261 matches in the gluten database (Table 4). Eighteen of these matches corresponded to an alpha-gliadin peptide that has previously been shown to be immunodominant in adult celiac disease patients, QLQPFPQPQLPY (Arentz-Hansen, 2000; Anderson, 2000).

Next we used the three algorithms to perform additional database searches in gluten-like storage molecules, in particular the hordeins from barley, the secalins from rye and the avenins from oats. While the first two cereals are toxic for CD patients, the latter is not (Janatuinen, E. K. et al., N. Engl. J. Med. 339: 1033-1087, 1995; Janatuinen. E. K. et al., Gut 46: 327-331, 2000). Strikingly, all three search algorithms yielded many matches in the available hordein and secalin sequences but none in the avenin sequences (Table 4). Consequently, the predicted T cell stimulatory gluten peptide QQPFPQQPQQPFPQ (FIG. 2) is present in hordeins and secalins but not in avenins (Table 4). Additional database searches with less strict algorithms revealed a general lack of matches in the avenin database as compared to the gluten, hordein and secalin databases (Table 4). Notably, no matches were found in the avenins with three variants of the predictive algorithm 2.

The enzyme pepsin is known to preferentially cleave proteins after phenylalanine and to a lesser extent after tyrosine, leucine, and isoleucine. Such a cleavage in the stomach will thus generate many gluten peptides with the tTG substrate sequence QXPF(Y) at the C-terminus. The subsequent activity of pepsin and tTG, therefore, favors the generation of many gluten peptides with an appropriate p7 anchor for binding to HLA-DQ2. We also demonstrate that by combining the HLA-DQ2 peptide binding motif with two deamidation patterns an algorithm is obtained that predicts novel T cell stimulatory peptides in gluten. Strikingly, in this predictive algorithm [X X X Q₄ X P₆ Q₇ X P₉ (Y,F,W,I,L)₁₀] we had incorporated a deamidation pattern that results in peptides that do not optimally fit the HLA-DQ2 peptide binding motif, e.g. they lack an amino acid with a large hydrophobic side chain at position 9 in the peptide. The lack of this anchor is probably compensated by the introduction of two negative charges in the peptide at p4 and p7 due to deamidation that serve as strong anchors. Thus, the T cell stimulatory activity of these peptides appears to depend more on optimal deamidation than on adherence to the exact HLA-DQ2 peptide binding motif. A less stringent search algorithm, combining only one deamidation rule with the HLA-DQ2 peptide binding motif proved equally successful since it predicted the previously identified T cell stimulatory alpha-gliadin peptide. Importantly, the first predictive algorithm identified 13 peptides, 8 out of which stimulated T cells. The second, less stringent algorithm predicted 261 peptides. Eighteen of those (1 in 15) matched a known T cell stimulatory gliadin peptide. These algorithms, therefore, have a high predictive value. It is striking, therefore, that these algorithms identified very similar and sometimes identical peptides in the barley and rye derived hordeins and secalins but not in the oats derived avenins. These results appear not related to the size of the avenin database since the secalin database is the smallest of the three investigated. Oats is considered safe or CD patients (Janatuinen, 1995; 2000). A striking difference between the amino acid content of gliadins, hordeins, and secalins on one side and the avenins on the other side is the relative lack of proline in the latter (Wieser, H. et al. Z. Lebensm. Unters. Forsch. 177: 457-460, 1983). While gliadins, hordeins and secalins contain approximately 86% glutamine and 20% proline, avenins contain a similar percentage of glutamine (84%) but half the amount of proline (10%). The lack of proline residues in Q-rich regions leads to non-selective deamidation of the glutamine residues in such regions. In the gluten sequence PFSQQQQPV, for example, the underlined Q-residues are specifically and efficiently deamidated by tTG while in the naturally occurring homologous sequence PFSQQQQLVL, in which the proline at p8 is replaced by a leucine, all Q-residues are deamidated. The deamidated peptide does not match the HLA-DQ2 peptide binding motif. Our results therefore offer a simple explanation for the clinical observation that oats is not toxic for CD patients. Moreover, our results indicate that the replacement of particular proline residues in gluten by other amino acids would yield gluten molecules with considerably less toxicity for CD patients but that would retain the chemical and physical properties that are so desired in gluten. Potentially this knowledge may be used to construct gluten molecules with lower toxicity or design oligonucleotide primers to identify wheat varieties that lack (some of) these relevant proline residues.

tTG Inhibitor/Blocker

A lead compound was designed on the basis of an octapeptide, sequence QPQLPYPQ, known to be a tTG substrate {Arentz-Hansen; H., et al., 2000} and containing the sequence QLPY which allows the transformation of the indicated glutamine (Q) into glutamate (Vader, L. W. et al. 2002). Based on the knowledge that tTG has a cysteine protease mode of action {Liu, S., R. A. Cerione, and J. Clardy. 2002. Structural basis for the guanine nucleotide-binding activity of tissue transglutaminase and its regulation of transamidation activity. Proc. Natl. Acad. Sci. U.S.A. 99:2748-2747}, we prepared with the aid of standard Fmoc-based solid-phase peptide synthesis, biotinylated QPQLPYQ in which the carboxamide moiety of the glutamine at position 3 in the substrate is replaced by a vinylogous ester as an electrophilic trap (CO₂Et, FIG. 3). The inhibitor was designed in such a way that manipulation and visualization could easily be performed in the studies; a biotin group was introduced to enable binding of the inhibitor to avidin/strepavidin in order to isolate/purify the inhibitor, the tyrosine is a possible iodination site to, label the peptide with a radio-label and a carboxy-terminus enables the site-specific coupling of amine-compounds of choice by amide bond formation It is clear that only the amino acid sequence of the inhibitor and the electrophilic trap in the side chain of the amino acid at position 3 are important parameters for inhibitory activity and that the other provided side groups are optional and are not important for the activity of the inhibitor/blocker.

The incorporation of the active site inhibitor in place of the glutamine residue was effected by coupling of a glutamic aldehyde species, protected as its dimethyl acetal. Upon acidic cleavage from the solid phase, the aldehyde was liberated and a Wittig reaction carried out on the crude peptide, yielding the target compound after HPLC purification.

This first-generation inhibitor was found to effectively inhibit the enzyme activity. When mixed with the enzyme in an equimolar ratio, the deamidation of a substrate peptide was inhibited for over 50%, at a 0.3 molar ratio of enzyme to inhibitor the enzyme activity was completely abolished, as demonstrated by mass spectrometric analysis of the substrate peptide as described {Vader, L. W, et al., 2002}. (FIG. 4).

TABLE 1
Deamidation patterns in the HLA-DQ8 restricted gliadin peptide.

| Pattern | Effect on doamidation |
|---|---|
| QP ↓ | no deamidation |
| QXX(F, Y, W, M, L, I, V) ↑ | good deamidation |
| QXXP ↓ | no deamidation |
| QXP ↑ | good deamidation |
| QXP(F, Y, W, M, L, I, V) ↑ | good deamidation |

| | | Rules | | | |
|---|---|---|---|---|---|
| | | | QXXF | QXXP | |
| Peptides | | QP ↓ | ↑ | ↓ | QXP ↑ |
| 1 | VPVP [symbol] LQPQNPSQ [symbol] QPQE [symbol] VPL | 100% | 100% | 100% | 66% |
| 2 | PLVQ [symbol] [symbol] QFLGQ [symbol] QPFPPQ | 100% | 100% | 100% | 100% |
| 3 | YYPTSP [symbol] QSGQG | — | — | — | — |
| 4 | QG [symbol] QGYYPTSPQ | — | 50% | — | — |
| 5 | SS [symbol] VSFQPSQLN | 100% | 100% | — | — |
| 6 | FPQT [symbol] QPQQLFPQSQ | 100% | 0% | 100% | 100% |
| 7 | G [symbol] QGYYPTSVQ [symbol] SGQ | — | 50% | — | — |
| 8 | GSVQP [symbol] Q [symbol] LPQFEIR | 100% | 100% | 100% | 100% |
| 9 | FPQHCNYQ [symbol] QPQTFPQPF | 100% | — | 100% | 100% |
| 10 | QQPIQPQ [symbol] FP [symbol] QQFF | 100% | 50% | 100% | 50% |
| 11 | QSG [symbol] YQ [symbol] QPQMQTT | 100% | — | 100% | 100% |
| 12 | ESQ [symbol] S [symbol] QDEPQPF | 100% | — | 100% | — |
| 13 | FPQPEDQ [symbol] SQQSE | 100% | — | — | — |
| 14 | LQQVQ [symbol] GP [symbol] [symbol] QPFPQPQPF | 100% | 100% | 66% | 100% |
| 15 | QVQWPQQQPFPQP [symbol] QPF | 100% | 50% | 100% | 33% |
| 16 | PLLP [symbol] QPFPS [symbol] QEQPQF | 100% | 50%\hat{ } | 100% | 100% |
| 17 | WQ [symbol] QPPFS [symbol] EQPIL | 100% | 0%\hat{ } | 33% | 100% |
| 18 | QSNILPQPA [symbol] QPFQP [symbol] VPQQP | 100% | 100% | — | 66% |
| 19 | WFQPSQLNP [symbol] A [symbol] QDQPQ | 100% | — | 100% | — |
| 20 | LQQP [symbol] QPQFQP [symbol] QQF | 100% | 50%\hat{ } | 100% | 50% |
| 21 | VQQ [symbol] IPVVQPSIL | 100% | 33%\hat{ } | 100% | 100% |

TABLE 3

Design of search algorithms based on the HLA-DQ2 peptide
binding motif and the tTG specificity.

| | | | | | | | | | | | | | | |
|---|---|---|---|---|---|---|---|---|---|---|---|---|---|---|
| Residue | −2 | −1 | 1 | 2 | 3 | 4 | 5 | 6 | 7 | 8 | 9 | 10 | 11 | 12 |

DQ2 peptide binding motif

| | | | | | | | | | | |
|---|---|---|---|---|---|---|---|---|---|---|
| Preferred | — | — | F,W, | D,E | P,A | D, | F,W,Y | — | — | — |
| | | | VI,L, | V,L | E | E | I,L,V, | | | |
| | | | V | I | | | M | | | |

DQ2 epitope selection algorithms

| | | | | | | | | | | | | | | |
|---|---|---|---|---|---|---|---|---|---|---|---|---|---|---|
| Algorithm | X | X | X | X | X | Q | X | P | Q | X | F,Y,W | X | X | X |
| 1 | | | | | | | | | | | I,L | | | |
| Algorithm | X | X | X | X | X | Q | X | P | Q | X | P | F,Y, | X | X |
| 2 | | | | | | | | | | | | WI,L | | |

Gluten Database search

| | | |
|---|---|---|
| Algorithm | Matches: 52 | Source: Gliadin (6); Glutenin (8) |
| 1 | Unique: 14 | |
| Algorithm | Matches: 46 | Source: Gliadin (all) |
| 2 | Unique 13 | |

TABLE 4
Database searches with the search algorithms.
The search algorithms reveal many matches in the gluten, hordeins and secalin
sequences but very few in the avenin sequences. The predictive algorithm 2
[QXPQXP(YFWIL)] and the less strict variants of that algorithm have no matches in
the avenin database. The novel T cells stimulatory gluten peptide
(QQPFPQQPQQPFPQ) is also found in the hordeins and secalin but not in the
avenins. Other search algorithms identify only a limited number of sequences in the
avenins.

| | | # of matches in databases | | | |
|---|---|---|---|---|---|
| Search algorithm | | gluten | hordein | secalin | avenin |
| Algorithm 1 | Q₄XPQ₇X(YFWIL) | 52 | 48 | 8 | — |
| Algorithm 1 - Q₄ | XXPQ₇X(YFWIL) | 334 | 91 | 14 | 6 |
| Algorithm 1 - Q₇ | Q₄XPXX(YFWIL) | 286 | 142 | 60 | 11 |
| Algorithm 2 | Q₄XPQ₇XP(YFWIL) | 46 | 60 | 33 | — |
| Algorithm 2 - Q₄* | XXPQ₇XP(YFWIL) | 261 | 196 | 89 | — |
| Algorithm 2 - Q₇ | Q₄XPXXP(YFWIL) | 51 | 68 | 33 | — |
| Short algorithm | QXP(YFWIL) X ≠ P** | >500 | 276 | 105 | 7 |
| Predicted epitope | QQPFPQQPQQPFPQ | 6 | 12 | 2 | — |

*Algorithm 2 without the specification of a Q at position 4 predicts the known gliadin α9 epitope
**A proline at p2 in this algorithm inhibits deamidation of the glutamine

TABLE 5
List of protein autoantigens in multiple sclerosis (MS), rheumatoid arthritis
(RA), diabetes (DB), systemic lupus erythematosus (SLE), Sjogren
syndrome (SS) and Coeliac disease (CD) and peptides derived from
these proteins that contain the tTG-substrate motif disclosed herein¹

| | Code | | Accession | Amino |
|---|---|---|---|---|
| Autoantigen^{2,5} | used³ | Database | number | acids⁴ |
| MS | | | | |
| Myelin basic protein | MBP | Swiss Prot | P02686 | 304 |
| Myelin-oligodendrocyte | MOG | Swiss Prot | Q16653 | 247 |
| glycoprotein | | | | |
| Myelin proteolipid protein | PLP | Swiss Prot | P06905 | 276 |
| RA | | | | |
| Complement C1q | C1QA | Swiss Prot | P02745 | 245 |
| subcomponent, A chain | | | | |
| Complement C1q | C1QB | Swiss Prot | P02746 | 251 |
| subcomponent, B chain | | | | |
| Complement C1q | C1QC | Swiss Prot | P02747 | 245 |
| subcomponent, C chain | | | | |
| Carbonic anhydrase II | CA2 | Swiss Prot | P00918 | 259 |
| Calpain inhibitor | CALP | Swiss Prot | P20810 | 708 |
| Alpha-1 type II collagen | COL2A1 | TrEMBL | Q14047 | 1487 |
| Filaggrin | FILA | Swiss Prot | P20930 | 416 |
| 78 kDa glucose- | GR78 | Swiss Prot | P11021 | 654 |
| regulated protein | | | | |
| Matrix metalloprotease-1 | MMP1 | Swiss Prot | P03956 | 469 |
| Matrix metalloprotease-3 | MMP3 | Swiss Prot | P08254 | 477 |
| Matrix metalloprotease-19 | MMP19 | Swiss Prot | Q99542 | 508 |
| Heterogenous nuclear | ROA2 | Swiss Prot | P22626 | 353 |
| ribonucleoproteins A2/B1 | | | | |
| Pulmonary surfactant- | SPA | Swiss Prot | P07714 | 248 |
| associated protein A | | | | |
| Vinculin | VINC | Swiss Prot | P18206 | 1065 |
| Chitinase 3-like protein 2 | YKL39 | Swiss Prot | Q15782 | 390 |
| Chitinase 3-like protein 1 | YKL40 | Swiss Prot | P36222 | 383 |
| DB | | | | |
| Carboxypeptidase H | CPH | Swiss Prot | P16870 | 476 |
| Delta-like protein | DLK | Swiss Prot | P80370 | 383 |
| Glutamate decarboxylase, | GAD | Swiss Prot | Q05329 | 585 |
| 65 kDa isoform | | | | |
| Glucose transporter type 4 | GLUT4 | Swiss Prot | P14672 | 509 |
| 60 KDa Heat shock protein | HSP60 | Swiss Prot | P10809 | 573 |
| Protein-tyrosine | ICA | Swiss Prot | Q16849 | 979 |
| phosphatase-like N | | | | |
| 69 kDa islet cell | ICA69 | Swiss Prot | Q05084 | 480 |
| autoantigen | | | | |
| Imogen 44 | IM44 | TrEMBL | Q61733 | 384 |
| Insulin precursor | INS | Swiss Prot | P01308 | 110 |
| Insulin receptor | INSR | Swiss Prot | P06213 | 1382 |
| Insulin receptor substrate-1 | IRS1 | Swiss Prot | P35568 | 1242 |
| SOX-13 protein | SOX13 | Swiss Prot | Q9UN79 | 889 |
| SLE/SS | | | | |
| Centromere protein A | CENPA | Swiss Prot | P49450 | 140 |
| Centromere protein B | CENPB | Swiss Prot | P07199 | 599 |
| DNA excision repair | ERCC1 | Swiss Prot | P07992 | 297 |
| protein ERCC-1 | | | | |
| 60S ribosomal protein L7 | RL7 | Swiss Prot | P18124 | 248 |
| 52 kDa ribonucleoprotein | ROSS | Swiss Prot | P27797 | 417 |
| autoantigen RO/SS-A | | | | |
| Small nuclear | SMPB | Swiss Prot | P14678 | 240 |
| ribonucleoprotein | | | | |
| associated | | | | |
| proteins B and B′ | | | | |
| Small nuclear | SMPN | Swiss Prot | P14648 | 240 |
| ribonucleoprotein | | | | |
| associated | | | | |
| protein N | | | | |
| SNRPB protein | SNRPB | TrEMBL | Q15182 | 285 |
| SSA protein SS-56 | SS56 | TrEMBL | Q96PF7 | 485 |
| Miscellaneous/CD | | | | |
| Tiisue transglutaminase | TTG | Swiss Prot | P21980 | 687 |
| ¹This table reflects the consensus amino acid sequences of the proteins mentioned. It is known that for some proteins mentioned variants exsist, like splice variants and variants having one or more amino acid mutations. These variants should be regarded as being | | | | |
| # comprised in the table. Also the peptides that are derived from these variant and that contain the tTG-substrate motif disclosed herein should be regarded as being comprised in this table. | | | | |
| ²Some of the proteins listed are also known by one or more other names | | | | |
| ³For some of the proteins listed one or more other codes are used in databases | | | | |
| ⁴Number of amino acids given here reflect the amino acid sequence indicated, numbers might be different for variants | | | | |
| ⁵Some antigens are known to possibly play a role in more then one of the diseases mentioned. The way the antigens are grouped is only meant for clarity and is by no means intended to limit the claims | | | | |
| ⁶Peptides are named by the sequence number in the protein that corresponds to the amino-terminal amino acid of the peptide | | | | |
| ⁷Only 19-mer peptides having the target Q at position 10 are shown. It is known that the length of HLA-bound peptides can vary from 8 to up to 40 amino acids. Longer peptides containing a 19-mer sequence or part of a 19-mer sequence from the table and shorter | | | | |
| # peptides containing part of a 19-mer sequence from the table are considered to be comprised in this table | | | | |

Multiple sclerosis (MS)

| MBP | |
|---|---|
| 246 | S R F S W G A E G Q R P G F G Y G G R |
| 272 | H K G F K G V D A Q G T L S K I F K L |
| MOG | |
| 22 | L Q V S S S Y A G Q F R V I G P R H P |
| 81 | R N G K D Q D G D Q A P E Y R G R T E |
| 159 | L A V L P V L L L Q I T V G L V F L C |
| 170 | T V G L V F L C L Q Y R L R G K L R A |
| PLP | |
| 59 | Y L I N V I H A F Q Y V I Y G T A S F |
| 224 | L S I C K T A E F Q M T F H L F I A A |

Rheumatoid arthritis (RA)

| C1QA | |
|---|---|
| 159 | Y Y F T F Q V L S Q W E I C L S I V S |
| 205 | M V L Q L Q Q G D Q V W V E K D P K K |
| C1QB | |
| 15 | L L L G L I D I S Q A Q L S C T G P P |
| 181 | N L M R G R E R A Q K V V T F C D Y A |
| C1QC | |
| 110 | E E G R Y K Q K F Q S V F T V T R Q T |
| CA2 | |
| 18 | D F P I A K G E R Q S P V D I D T H T |
| 82 | P L D G T Y R L I Q F H F H W G S L D |
| 125 | K Y G D F G K A V Q Q P D G L A V L G |
| 147 | K V G S A K P G L Q K V V D V L D S I |
| CALP | |
| 81 | K K A V S R S A E Q Q P S E K S T E P |
| 95 | K S T E P K T K P Q D M I S A G G E S |
| 536 | A A A I S E V V S Q T P A S T T Q A G |
| 639 | E D S K K P A D D Q D P I D A L S G D |
| COL2A1 | |
| 116 | V G P K G P P G P Q G P A G E Q G P R |
| 122 | P G P Q G P A G E Q G P R G D R G D K |
| 186 | G F D E K A G G A Q L G V M Q G P M G |
| 191 | A G G A Q L G V M Q G P M G P M G P R |
| 380 | T G A R G P E G A Q G P R G E P G T P |
| 458 | P G I A G F K G E Q G P K G E P G P A |
| 511 | A P G N R G F P G Q D G L A G P K G A |
| 563 | T G R P G D A G P Q G K V G P S G A P |
| 707 | R G S P G A Q G L Q G P R G L P G T P |
| 857 | K G D A G A P G P Q G P S G A P G P Q |
| 866 | Q G P S G A P G P Q G P T G V T G P K |
| 938 | P G R A G E P G L Q G P A G P P G E K |
| 970 | P P G P Q G L A G Q R G I V G L P G Q |
| 1085 | Q G D R G E A G A Q G P M G P S G P A |
| 1100 | S G P A G A R G I Q G P Q G P R G D K |
| 1103 | A G A R G I Q G P Q G P R G D K G E A |
| 1365 | N L A P N T A N V Q M T F L R L L S T |
| FILA | |
| 62 | S R H S T S Q E G Q D T I H G H R G S |
| 199 | A S R N H H G S A Q E Q L R D G S R H |
| 386 | S R H S A S Q D G Q D T I R G H P G S |
| GR78 | |
| 100 | G R T W N D P S V Q Q D I K F L P F K |
| 120 | V E K K T K P Y I Q V D I G G G Q T K |
| 251 | T H L G G E D F D Q R V M E H F I K L |
| 295 | A K R A L S S Q H Q A R I E I E S F Y |
| 334 | L F R S T M K P V Q K V L E D S D L K |
| 363 | G G S T R I P K I Q Q L V K E F F N G |
| 392 | E A V A Y G A A V Q A G V L S G D Q D |
| 487 | I P P A P R G V P Q I E V T F E I D V |
| 521 | K N K I T I T N D Q N R L T P E E I E |
| MMP1 | |
| 26 | T Q E Q D V D L V Q K Y L E K Y Y N L |
| 59 | P V V S K L K Q M Q E F F G L K V T G |
| 90 | P R C G V P D V A Q F V L T E G N P R |
| 102 | L T E G N P R W E Q T H L T Y R I E N |
| 147 | L T F T K V S E G Q A D I M I S F V R |
| 241 | T F S G D V Q L A Q D D I D G I Q A I |
| 248 | L A Q D D I D G I Q A I Y G R S Q N P |
| 255 | G I Q A I Y G R S Q N P V Q P I G P Q |
| 264 | Q N P V Q P I G P Q T P K A C D S K L |
| 314 | L N F I S V F W P Q L P N G L E A A Y |
| 345 | G N K Y W A V Q G Q N V L H G Y P K D |
| 435 | F F Y F F H G T R Q Y K F D P K T K R |
| MMP3 | |
| 26 | G E D T S M N L V Q K Y L E N Y Y D L |
| 59 | P V V K K I R E M Q K F L G L E V T G |
| 244 | T D L T R F R L S Q D D I N G I Q S L |
| 251 | L S Q D D I N G I Q S L Y G P P P D S |
| 446 | F F Y F F T G S S Q L E F D P N A K K |
| MMP19 | |
| 25 | E V A P V D Y L S Q Y G Y L Q K P L E |
| 30 | D Y L S Q Y G Y L Q K P L E G S N N F |
| 84 | R C G L E D P F N Q K T L K Y L L L G |
| 153 | D I R L S F H G R Q S S Y C S N T F D |
| 218 | L G L G H S R Y S Q A L M A P V Y E G |
| 243 | L H P D D V A G I Q A L Y G K K S P V |
| 381 | D A A L Y W P L N Q K V F L F K G S G |
| 445 | G K V Y W R L N Q Q L R V C K G Y P R |
| ROA2 | |
| 299 | N Y N D F G N Y N Q Q P S N Y G P M K |
| SPA | |
| 99 | L P A H L D E E L Q A T L H D F R H Q |
| 119 | L Q T R G A L S L Q G S I M T V G E K |
| VINC | |
| 169 | K M A K M I D E R Q Q E L T H Q E H R |
| 382 | S I A K K I D A A Q N W L A D P N G G |
| 491 | H L E G K I E Q A Q R W I D N P T V D |
| 506 | P T V D D R G V G Q A A I R G L V A E |
| 529 | A N V M M G P Y R Q D L L A K C D R V |
| 567 | Q A R A L A S Q L Q D S L K D L K A R |
| 679 | R I L L R N P G N Q A A Y E H F E T M |
| 737 | K V A M A N I Q P Q M L V A G A T S I |
| 804 | A G N I S D P G L Q K S F L D S G Y R |
| 828 | A K V R E A F Q P Q E P D F P P P P P |
| 841 | F P P P P P D L E Q L R L T D E L A P |
| 984 | C E R I P T I S T Q L K I L S T V K A |
| 1018 | A T E M L V H N A Q N L M Q S V K E T |
| YKL39 | |
| 32 | Y F T N W S Q D R Q E P G K F T P E N |
| 158 | L I H E L A E A F Q K D F T K S T K E |
| 314 | A K I T R L Q D Q Q V P Y A V K G N Q |
| 365 | D D F T G K S C N Q G P Y P L V Q A V |
| YKL40 | |
| 162 | I K E A Q P G K K Q L L L S A A L S A |
| 306 | A T V H R T L G Q Q V P Y A T K G N Q |
| 322 | G N Q W V G Y D D Q E S V K S K V Q Y |
| 351 | V W A L D L D D F Q G S F C G Q D L R |

Diabetes (DB)

| CPH | |
|---|---|
| 19 | C G W L L G A E A Q E P G A P A A G M |
| DLK | |
| 24 | A E C F P A C N P Q N G F C E D D N V |
| 41 | N V C R C Q P G W Q G P L C D Q C V T |
| 219 | N G G T C L Q H T Q V S Y E C L C K P |
| 269 | T P G V H S L P V Q Q P E H R I L K V |
| GAD | |
| 68 | A R K A A C A C D Q K P C S C S K V D |
| 103 | G E R P T L A F L Q D V M N I L L Q Y |
| 111 | L Q D V M N I L L Q Y V V K S F D R S |
| 155 | N L E E I L M H C Q T T L K Y A I K T |
| 315 | L E R R I L E A K Q K G F V P F L V S |
| GLUT4 | |
| 8 | I G S E D G E P P Q Q R V T G T L V L |
| 28 | V F S A V L G S L Q F G Y N I G V I N |
| 40 | Y N I G V I N A P Q K V I E Q S Y N E |
| 168 | L R G A L G T L N Q L A I V I G I L I |
| 207 | G L T V L P A L L Q L V L L P F C P E |
| 404 | W F I V A E L F S Q G P R P A A M A V |
| HSP60 | |
| 231 | T S K G Q K C E F Q D A Y V L L S E K |
| 246 | L S E K K I S S I Q S I V P A L E I A |
| 363 | D K A Q I E K R I Q E I I E Q L D V T |
| 368 | E K R I Q E I I E Q L D V T T S E Y E |
| ICA | |
| 46 | L C S H L E V C I Q D G L F G Q C Q V |
| 52 | V C I Q D G L F G Q C Q V G V G Q A R |
| 54 | I Q D G L F G Q C Q V G V G Q A R P L |
| 75 | V T S P V L Q R L Q G V L R Q L M S Q |
| 94 | G L S W H D D L T Q Y V I S Q E M E R |
| 145 | I P T G S A P A A Q H R L P Q P P V G |
| 168 | G A S S S L S P L Q A E L L P P L L E |
| 469 | E E Y G Y I V T D Q K P L S L A A G V |
| 524 | N L S L A D V T Q Q A G L V K S E L E |
| 535 | G L V K S E L E A Q T G L Q I L Q T G |
| 542 | E A Q T G L Q I L Q T G V G Q R E E A |
| 675 | T P S W C E E P A Q A N M D I S T G H |
| 775 | P R M P A Y I A T Q G P L S H T I A D |
| 787 | L S H T I A D F W Q M V W E S G C T V |
| 860 | Q T Q E T R T L T Q F H F L S W P A E |
| 939 | A A T L E H V R D Q R P G L V R S K D |
| 949 | R P G L V R S K D Q F E F A L T A V A |
| ICA69 | |
| 4 | H K C S Y P W D L Q D R Y A Q D K S V |
| 18 | Q D K S V V N K M Q Q R Y W E T K Q A |
| 19 | D K S V V N K M Q Q R Y W E T K Q A F |
| 26 | M Q Q R Y W E T K Q A F I K A T G K K |
| 72 | D L S K A I V L Y Q K R I C F L S Q E |
| 116 | T G K A L C F S S Q Q R L A L R N P L |
| 186 | K Q M E K F R K V Q T Q V R L A K K N |
| 188 | M E K F R K V Q T Q V R L A K K N F D |
| 226 | L L S H M L A T Y Q T T L L H F W E K |
| 262 | Y E F T T L K S L Q D P M K K L V E K |
| 288 | Q Q E S T D A A V Q E P S Q L I S L E |
| 430 | L L D Q N M K D L Q A S L Q E P A K A |
| 434 | N M K D L Q A S L Q E P A K A A S D L |
| IM44 | |
| 37 | G A V R T E N N I Q R H F C T S R S I |
| 203 | P S G R A S T R P Q H Q I Q F D E D M |
| 205 | G R A S T R P Q H Q I Q F D E D M D S |
| 324 | D K Y L E D F P K Q G P I R L F M E L |
| INS | |
| 56 | R E A E D L Q V G Q V E L G G G P G A |
| INSR | |
| 52 | N C S V I E G H L Q I L L N F K T R P |
| 294 | K N S R R Q G C H Q Y V I H N N K C I |
| 441 | W S K H N L T T T Q G K L F P H Y N P |
| 579 | L M R G L K P W T Q Y A I F V K T L V |
| 628 | P I S V S N S S S Q I I L K W K P P S |
| 690 | E S E D S Q K H N Q S E Y E D S A G E |
| 863 | E N N V V H L M W Q E P K E P N G L I |
| 1022 | K I T L L R E L G Q G S F G M V Y E G |
| 1125 | N P G R P P P T L Q E M I Q M A A E I |
| IRS1 | |
| 94 | A I A A D S E A E Q D S W Y Q A L L Q |
| 99 | S E A E Q D S W Y Q A L L Q L H N R A |
| 156 | P G P A F K E V W Q V I L K P K G L G |
| 287 | H H L N N P P P S Q V G L T R R S R T |
| 533 | T I T H Q K T P S Q S S V A S I E E Y |
| 635 | M S P K S V S A P Q Q I I N P I R R H |
| 766 | S L P R S F K H T Q R P G E P E E G A |
| 872 | P R A R E Q Q Q Q Q Q P L L N P P E P |
| 896 | Y V N I E F G S D Q S G Y L S G P V A |
| 990 | M T M Q M S C P R Q S Y V D T S P A A |
| 1055 | A H S S L L G G P Q G P G G M S A F T |
| 1181 | D L D L V K D F K Q C P Q E C T P E P |
| SOX13 | |
| 35 | R A S Q D S A D P Q A P A Q G N F R G |
| 39 | D S A D P Q A P A Q G N F R G S W D C |
| 72 | G V S E A A S G S Q E K L D F N R N L |
| 115 | M E A K D V K G T Q E S L A E K E L Q |
| 124 | Q E S L A E K E L Q L L V M I H Q L S |
| 146 | D Q L L T A H S E Q K N M A A M L F E |
| 158 | M A A M L F E K Q Q Q Q M E L A R Q Q |
| 160 | A M L F E K Q Q Q Q M E L A R Q Q Q E |
| 168 | Q Q M E L A R Q Q Q E Q I A K Q Q Q Q |
| 175 | Q Q Q E Q I A K Q Q Q Q L I Q Q Q H K |
| 176 | Q Q S Q I A K Q Q Q Q L I Q Q Q H K I |
| 182 | K Q Q Q Q L I Q Q Q H K I N L L Q Q Q |
| 189 | Q Q Q H K I N L L Q Q Q I Q Q V N M P |
| 194 | I N L L Q Q Q I Q Q V N M P Y V M I P |
| 219 | Q P L P V T P D S Q L A L P I Q P I P |
| 260 | G A M A T H H P L Q E P S Q P L N L T |
| 450 | Q E K Q P Y Y E E Q A R L S R Q H L E |
| 499 | M R T R R Q D A R Q S Y V I P P Q A G |
| 509 | S Y V I P P Q A G Q V Q M S S S D V L |
| 685 | S T S A F R A Y G Q G T L Y D S P L L |
| 695 | G T L Y D S P L L Q V S I H L G Y G I |
| 750 | I T R I A L Y F V Q K G L A V P C C F |
| 856 | L S L S V L V S L Q G P L F L S Y L G |

Systemic lupus erythematosus (SLE) and Sjogren
syndrome (SS)

| | |
|---|---|
| CENPA | |
| 36 | S S H Q H S R R R Q G W L K E I R K L |
| 80 | R G V D F N W Q A Q A L L A L Q E A A |
| CENPE | |
| 180 | P S V A E G Y A S Q D V F S A T E T S |
| 245 | G K S A K P R A G Q A G L P C D Y T A |
| 304 | L D T S G L R H V Q L A F F P P G T V |
| 330 | V Q Q V K G H Y R Q A M L L K A M A A |
| 343 | L K A M A A L E G Q D P S G L Q L G L |
| 349 | L E G Q D P S G L Q L G L T E A L H F |
| 469 | E G L E A E D W A Q G V V E A G G S F |
| 488 | G A Y G A Q E E A Q C P T L H F L E G |
| 559 | T S F P I D D R V Q S H I L H L E H D |
| 580 | H V T R K N H A R Q A G V R G L G H Q |
| ERCC1 | |
| 163 | N F A L R V L L V Q V D V K D P Q Q A |
| 170 | L V Q V D V K D P Q Q A L K E L A K M |
| 208 | Y L E T Y K A Y E Q K P A D L L M E K |
| 220 | A D L L M E K L E Q D F V S R V T E C |
| 242 | V K S V N K T D S Q T L L T T F G S L |
| RL7 | |
| 30 | I K R L R K K F A Q K M L R K A R R K |
| ROSS | |
| 80 | S F E P F S N K G Q T L V V Q F T V K |
| 85 | S N K G Q T L V V Q F T V K H E Q N I |
| 110 | V K L F P N S L D Q T D M H G D S E Y |
| SMPB | |
| 113 | G I P A G V P M P Q A P A G L A G P V |
| 130 | P V R G V G G P S Q Q V M T P Q G R G |
| SMPN | |
| 113 | G V P A G V P I P Q A P A G L A G P V |
| 130 | P V R G V G G P S Q Q V M T P Q G R G |
| SNRPB | |
| 113 | G I P A G V P M P Q A P A G L A G P V |
| 130 | P V R G V G G P S Q Q V M T P Q G R G |
| SS56 | |
| 113 | V L I M C E A C S Q S P E H E A H S V |
| 173 | W K I Q V E T R K Q S I V W E F E K Y |
| 183 | S I V W E F E K Y Q R L L S K K Q P P |
| 228 | E L N H S E L I Q Q S Q V L W R M I A |
| 230 | N H S S L I Q Q S Q V L W R M I A E L |
| 244 | M I A E L K E R S Q R P V R W M L Q D |
| 255 | P V R W H L Q D I Q E V L N R S K S W |
| 267 | L N R S K S W S L Q Q P E P I S L E L |

Miscellaneous/Celiac disease

TTG

| | |
|---|---|
| 42 | T L H F E G R N Y Q A S V D S L T F S |
| 148 | V Y L D S E E E R Q E Y V L T Q Q G F |
| 154 | E E R Q E Y V L T Q Q G F I Y Q G S A |
| 155 | E R Q E Y V L T Q Q G F I Y Q G S A K |
| 177 | N I P W N F G Q F Q D G I L D I C L I |
| 225 | S G M V N C N D D Q G V L L G R W D N |
| 267 | H G C Q R V K Y G Q C W V F A A V A C |
| 339 | E S W M T R P D L Q P G Y E G W Q A L |
| 601 | R K L V A E V S L Q N P L P V A L E G |
| 624 | V E G A G L T E E Q K T V E I P D P V |

1. Peptide sequence of 14-40 amino acids, which is prone to deamidation by tTG and which is a causative factor of celiac disease, which comprises an amino acid sequence selected from the group consisting essentially of QQPYPQQPQQPFPQ, QQPFPQQPQQPFPQ, PFPQTQQPQQPFPQ, PFPQLQQPQQPFPQ, QFPQTQQPQQPFPQ, QLPFPQQPQQPFPQ, PFPQPQQPQQPRPQ and PFPQSQQPQPPFPQ.

2. Epitope which is the causative factor of celiac disease, selected from the group consisting essentially of QQPYPEQPQQPFPQ, QQPFPEQPQQPFPQ, PFPQTEQPQQPFPQ, PFPQLEQPQQPFPQ, QFPQTEQPQQPFPQ, QLPFPEQPQQPFPQ, PFPQPEQPQQPFPQ, PFPQSEQPQQPFPQ, QQPYPEQPEQPFPQ, QQPFPEQPEQPFPQ, PFPQTEQPEQPFPQ, PFPQLEQPEQPFPQ, QFPQTEQPEQPFPQ, QLPFPEQPEQPFPQ, PFPQPEQPEQPFPQ, PFPQSEQPEQPFPQ, QQPYPQQPEQPFPQ, QQPFPQQPEQPFPQ, PFPQTQQPEQPFPQ, PFPQLQQPEQPFPQ, QFPQTQQPEQPFPQ, QLPFPQQPEQPFPQ, PFPQPQQPEQPFPQ and PFPQSQQPEQPFPQ, wherein further Q residues may be deamidated into an E residue.

3. Peptide sequence, which is prone to deamidation by tTG and which is a causative factor of an autoimmune disease selected from the group consisting essentially of RA (rheumatoid arthritis), MS (multiple sclerosis), SLE (systemic lupus erythomatosus), SS (Sjogren syndrome) and DB (diabetes), selected from the group consisting essentially of:
S R F S W G A E G Q R P G F G Y G G R,
H K G F K G V D A Q G T L S K I F K L,
L Q V S S S Y A G Q F R V I G P R H P,
R N G K D Q D G D Q A P E Y R G R T E,
L A V L P V L L L Q I T V G L V F L C,
T V G L V F L C L Q Y R L R G K L R A,
Y L I N V I H A F Q Y V I Y G T A S F,
L S I C K T A E F Q M T F H L F I A A,
Y Y F T F Q V L S Q W E I C L S I V S,
M V L Q L Q Q G D Q V W V E K D P K K,
L L L G L I D I S Q A Q L S C T G P P,
N L M R G R E R A Q K V V T F C D Y A,
E E G R Y K Q K F Q S V F T V T R Q T,
D F P I A K G E R Q S P V D I D T H T,
P L D G T Y R L I Q F H F H W G S L D,
K Y G D F G K A V Q Q P D G L A V L G,
K V G S A K P G L Q K V V D V L D S I,
K K A V S R S A E Q Q P S E K S T E P,
K S T E P K T K P Q D M I S A G G E S,
A A A I S E V V S Q T P A S T T Q A G,
E D S K K P A D D Q D P I D A L S G D,
V G P K G P P G P Q G P A G E Q G P R,
P G P Q G P A G E Q G P R G D R G D K,
G F D E K A G G A Q L G V M Q G P M G,
A G G A Q L G V H Q G P M G P M G P R,
T G A R G P E G A Q G P R G E P G T P,
P G I A G F K G E Q G P K G E P G P A,
A P G N R G F P G Q D G L A G P K G A,
T G R P G D A G P Q G K V G P S G A P,
R G S P G A Q G L Q G P R G L P G T P,
K G D A G A P G P Q G P S G A P G P Q,
Q G P S G A P G P Q G P T G V T G P K,
P G R A G E P G L Q G P A G P P G E K,
P P G P Q G L A G Q R G I V G L P G Q,
Q G D R G E A G A Q G P M G P S G P A,
S G P A G A R G I Q G P Q G P R G D K,
A G A R G I Q G P Q G P R G D K G E A,
N L A P N T A N V Q M T F L R L L S T,
S R H S T S Q E G Q D T I H G H R G S,
A S R N H H G S A Q E Q L R D G S R H,
S R H S A S Q D G Q D T I R G H P G S,
G R T W N D P S V Q Q D I K F L P F K,
V E K K T K P Y I Q V D I G G G Q T K,
T H L G G E D F D Q R V M E H F I K L,
A K R A L S S Q H Q A R I E I E S F Y,
L F R S T M K P V Q K V L E D S D L K,
G G S T R I P K I Q Q L V K E F F N G,
E A V A Y G A A V Q A G V L S G D Q D,
I P P A P R G V P Q I E V T F E I D V,
K N K I T I T N D Q N R L T P E E I E,
T Q E Q D V D L V Q K Y L E K Y Y N L,
P V V E K L K Q M Q E F F G L K V T G,
P R C G V P D V A Q F V L T E G N P R,
L T E G N P R W E Q T H L T Y R I E N,
L T F T K V S E G Q A D I M I S F V R,
T F S G D V Q L A Q D D I D G I Q A I,
L A Q D D I D G I Q A I Y G R S Q N P,
G I Q A I Y G R S Q N P V Q P I G P Q,
Q N P V Q P I G P Q T P K A C D S K L,
L N F I S V F W P Q L P N G L E A A Y,
G N K Y W A V Q G Q N V L H G Y P K D,
F F Y F F H G T R Q Y K F D P K T K R,
G E D T S M N L V Q K Y L E N Y Y D L,
P V V K K I R E M Q K F L G L E V T G,
T D L T R F R L S Q D D I N G I Q S L,
L S Q D D I N G I Q S L Y G P P P D S,
F F Y F F T G S S Q L E F D P N A K K,
E V A P V D Y L S Q Y G Y L Q K P L E,
D Y L S Q Y G Y L Q K P L E G S N N F,
R C G L E D P F N Q K T L K Y L L L G,
D I R L S F H G R Q S S Y C S N T F D,
L G L G H S R Y S Q A L M A P V Y E G,
L H P D D V A G I Q A L Y G K K S P V,
D A A L Y W P L N Q K V F L F K G S G,
G K V Y W R L N Q Q L R V E K G Y P R,
N Y N D F G N Y N Q Q P S N Y G P M K,
L P A H L D E E L Q A T L H D F R H Q,
L Q T R G A L S L Q G S I M T V G E K,
K M A K M I D E R Q Q E L T H Q E H R,
S I A K K I D A A Q N W L A D P N G G,
H L E G K I E Q A Q R W I D N P T V D,
P T V D D R G V G Q A A I R G L V A E,
A N V M M G P Y R Q D L L A K C D R V,
Q A R A L A S Q L Q D S L K D L K A R,
R I L L R N P G N Q A A Y E H F E T M,
K V A M A N I Q P Q M L V A G A T S I,
A G N I S D P G L Q K S F L D S G Y R,
A K V R E A F Q P Q E P D F P P P P P,
F P P P P P D L E Q L R L T D E L A P,
C E R I P T I S T Q L K I L S T V K A,
A T E M L V H N A Q N L M Q S V K E T,
Y F T N W S Q D R Q E P G K F T P E N,
L I H E L A E A F Q K D F T K S T K E,
A K I T R L Q D Q Q V P Y A V K G N Q,
D D F T G K S C N Q G P Y P L V Q A V,
I K E A Q P G K K Q L L L S A A L S A,
A T V H R T L G Q Q V P Y A T K G N Q,
G N Q W V G Y D D Q E S V K S K V Q Y,
V W A L D L D D F Q G S F C G Q D L R,
C G W L L G A E A Q E P G A P A A G M,
A E C F P A C N P Q N G F C E D D N V,
N V C R C Q P G W Q G P L C D Q C V T,
N G G T C L Q H T Q V S Y E C L C K P,
T P G V H E L P V Q Q P E H R I L K V,
A R K A A C A C D Q K P C S C S K V D,
G E R P T L A F L Q D V M N I L L Q Y,
L Q D V M N I L L Q Y V V K S F D R S,
N L E E I L M H C Q T T L K Y A I K T,
L E R R I L E A K Q K G F V P F L V S,
I G S E D G E P P Q Q R V T G T L V L,
V F S A V L G S L Q F G Y N I G V I N,
Y N I G V I N A P Q K V I E Q S Y N E,
L R G A L G T L N Q L A I V I G I L I,
G L T V L P A L L Q L V L L P F C P E,
W F I V A E L F S Q G P R P A A M A V,
T S K G Q K C E F Q D A Y V L L S E K,
L S E K K I S S I Q S I V P A L E I A,
D K A Q I E K R I Q E I I E Q L D V T,
E K R I Q E I I E Q L D V T T S E Y E,
L C S H L E V C I Q D G L F G Q C Q V,
V C I Q D G L F G Q C Q V G V G Q A R,
I Q D G L F G Q C Q V G V G Q A R P L,
V T S P V L Q R L Q G V L R Q L M S Q,
G L S W H D D L T Q Y V I S Q E M E R,
I P T G S A P A A Q H R L P Q P P V G,
G A S S S L S P L Q A E L L P P L L E,
E E Y G Y I V T D Q K P L S L A A G V,
N L S L A D V T Q Q A G L V K S E L E,
G L V K S E L E A Q T G L Q I L Q T G,
E A Q T G L Q I L Q T G V G Q R E E A,
T P S W C E E P A Q A N M D I S T G H,
P R M P A Y I A T Q G P L S H T I A D,
L S H T I A D F W Q N V W E S G C T V,
Q T Q E T R T L T Q F H F L S W P A E,
A A T L E H V R D Q R P G L V R S K D,
R P G L V R S K D Q F E F A L T A V A,
H K C S Y P W D L Q D R Y A Q D K S V,
Q D K S V V N K M Q Q R Y W E T K Q A,
D K S V V N K M Q Q R Y W E T K Q A F,
M Q Q R Y W E T K Q A F I K A T G K K,
D L S K A I V L Y Q K R I C F L S Q E,
T G K A L C F S S Q Q R L A L R N P L,
K Q M E K F R K V Q T Q V R L A K K N,
M E K F R K V Q T Q V R L A K K N F D,
L L S H M L A T Y Q T T L L H F W E K,
Y E F T T L K S L Q D P M K K L V E K,
Q Q E S T D A A V Q E P S Q L I S L E,
L L D Q N M K D L Q A S L Q E P A K A,
N M K D L Q A S L Q E P A K A A S D L,
G A V R T E N N I Q R H F C T S R S I,
P S G R A S T R P Q H Q I Q F D E D M,
G R A S T R P Q H Q I Q F D E D M D S,
D K Y L E D F P K Q G P I R L F M E L,
R E A E D L Q V G Q V E L G G G P G A,
N C S V I E G H L Q I L L M F K T R P,
K N S R R Q G C H Q Y V I H N N K C I,
W S K H N L T T T Q G K L F F H Y N P,
L M R G L K P W T Q Y A I F V K T L V,
P I S V S N S S S Q I I L K W K P P S,
E S E D S Q K H N Q S E Y E D S A G E,
E N N V V H L M W Q E P K E P N G L I,
K I T L L R E L G Q G S F G M V Y E G,
N P G R P P P T L Q E M I Q M A A E I,
A I A A D S E A E Q D S W Y Q A L L Q,
S E A E Q D S W Y Q A L L Q L H N R A,
P G P A F K E V W Q V I L K P K G L G,
H H L N N P P P S Q V G L T R R S R T,
T I T H Q K T P S Q S S V A S I E E Y,
M S P K S V S A P Q Q I I N P I R R H,
S L P R S F K H T Q R P G E P E E G A,
P R A R E Q Q Q Q Q Q P L L H P P E P,
Y V N I E F G S D Q S G Y L S G P V A,
M T M Q M S C P R Q S Y V D T S P A A,
A H S S L L G G P Q G P G G M S A F T,
D L D L V K D F K Q C P Q E C T P E P,
R A S Q D S A D P Q A P A Q G N F R G,
D S A D P Q A P A Q G N F R G S W D C,
G V S E A A S G S Q E K L D F N R N L,
M E A K D V K G T Q E S L A E K E L Q,
Q E S L A E K E L Q L L V M I H Q L S,
D Q L L T A H S E Q K N M A A M L F E,
M A A M L F E K Q Q Q Q M E L A R Q Q,
A M L F E K Q Q Q Q M E L A R Q Q Q E,
Q Q M E L A R Q Q Q E Q I A K Q Q Q Q,
Q Q Q E Q I A K Q Q Q Q L I Q Q Q H K,
Q Q E Q I A K Q Q Q Q L I Q Q Q H K I,
K Q Q Q Q L I Q Q Q H K I N L L Q Q Q,
Q Q Q H K I N L L Q Q Q I Q Q V N M P,
I N L L Q Q Q I Q Q V N M P Y V M I P,
Q P L P V T P D S Q L A L P I Q P I P,
G A M A T H H P L Q E P S Q P L N L T,
Q E K Q P Y Y E E Q A R L S R Q H L E,
M R T R R Q D A R Q S Y V I P P Q A G,
S Y V I P P Q A G Q V Q M S S S D V L,
S T S A F R A Y G Q G T L Y D S P L L,
G T L Y D S P L L Q V S I H L G Y G I,
I T R I A L Y F V Q K G L A V P C C F,
L S L S V L V S L Q G P L F L S Y L G,
S S H Q H S R R R Q G W L K E I R K L,
R G V D F N W Q A Q A L L A L Q E A A,
P S V A E G Y A S Q D V F S A T E T S,
G K S A K P R A G Q A G L P C D Y T A,
L D T S G L R H V Q L A F F P P G T V,
V Q Q V K G H Y R Q A M L L K A M A A,
L K A M A A L E G Q D P S G L Q L G L,
L E G Q D P S G L Q L G L T E A L H F,
E G L E A E D W A Q G V V E A G G S F,
G A Y G A Q E E A Q C P T L H F L E G,
T S F P I D D R V Q S H I L H L E H D,
H V T R K N H A R Q A G V R G L G H Q,
N F A L R V L L V Q V D V K D P Q Q A,
L V Q V D V K D P Q Q A L K E L A K M,
Y L E T Y K A Y E Q K P A D L L M E K,
A D L L M E K L E Q D F V S R V T E C,
V K S V N K T D S Q T L L T T F G S L,
I K R L R K K F A Q K M L R K A R R K,
S F E P F S N K G Q T L V V Q F T V K,
S N K G Q T L V V Q F T V K H E Q N I,
V K L F P N S L D Q T D M H G D S E Y,
G I P A G V P M P Q A P A G L A G P V,
P V R G V G G P S Q Q V M T P Q G R G,
G V P A G V P I P Q A P A G L A G P V,
P V R G V G G P S Q Q V M T P Q G R G,
G I P A G V P M P Q A P A G L A G P V,
P V R G V G G P S Q Q V M T P Q G R G,
V L I M C E A C S Q S P E H E A H S V,
W K I Q V E T R K Q S I V W E F E K Y,
S I V W E F E K Y Q R L L E K K Q P P,
E L N H S E L I Q Q S Q V L W R M I A,
N H S E L I Q Q S Q V L W R M I A E L,
M I A E L K E R S Q R P V R W M L Q D,
P V R W M L Q D I Q E V L N R S K S W,
L N R S K S W S L Q Q P E P I S L E L,
T L H F E G R N Y Q A S V D S L T F S,
V Y L D S E E E R Q E Y V L T Q Q G F,
E E R Q E Y V L T Q Q G F I Y Q G S A,
E R Q E Y V L T Q Q G F I Y Q G S A K,
N I P W N F G Q F Q D G I L D I C L I,
S G M V N C N D D Q G V L L G R W D N,
H G C Q R V K Y G Q C W V F A A V A C,
E S W M T R P D L Q P G Y E G W Q A L,
R K L V A E V S L Q N P L P V A L E G,
and
V E G A G L T E E Q K T V E I P D P V.

4. Peptide sequence of 8-40 amino acid residues which comprises at least one 8-mer which is a subsequence of at least one of the sequences listed in claim 3, wherein said 8-mer comprises the central Q residue of the sequence listed in claim 3.

5. An epitope formed by deamidation of one or more of the Q residues of the peptide according to claim 3.

6. An isolated HLA-DQ restricted T-cell capable of recognising an epitope according to claim 2.

7. An isolated or recombinant HLA-DQ restricted T-cell receptor capable of recognising an epitope according to claim 2.

8. An antibody reactive with an epitope according to claim 2.

9. A method to screen foodstuffs or plant material for the occurrence of an epitope according to claim 2 using a T cell, a T cell receptor or an antibody capable of recognising or binding to the epitope of claim 2.

10. Method according to claim 9, wherein the foodstuff or plant material is pretreated with a protease.

11. Method according to claim 10, wherein the protease is trypsin, chymotrypsin or pepsin.

12. Method according to claim 11, wherein the foodstuff or plant material is additionally treated with tTG.

13. A diagnostic kit comprising:
a T cell, a T cell receptor or an antibody capable of binding to an epitope in accordance with claim 2; and
a suitable means of detection.

14. A diagnostic kit comprising a T cell, a T cell receptor or an antibody capable of detecting the presence of a peptide according to claim 1.

15. An oligonucleotide primer set suitable for use in an amplification assay which is capable of binding to a nucleotide sequence coding for a peptide which matches a consensus sequence according to the following formula (1), (2), (3), (4) or (5):
X₁-X₂-X₃-Q₄-X₅-P₆-Q₇-X₈-P₉-X₁₀ (1)
in which X₁₀ is F, Y, M, W, I or L and X₁, X₂, X₃, X₅, and X₈ are any amino acid;
X₁-X₂-X₃-Q₄-X₅-P₆-Q₇-X₈-X₉-X₁₀ (2)
in which X₉ is F, Y, M, W, I or L and X₁, X₂, X₃, X₅, X₈ and X₁₀ are any amino acid;
Q-X₁-P (3)
in which X₁ is any amino acid;
Q-X₁-P-X₂ (4)
in which X₁ is any amino acid and X₂ is F,Y,W,M,L,I, or V;
Q-X₁-X₂-X₃ (5)
in which X₁ and X₂ are any amino acid and X₃ is F,Y,W,M,L,I or V.

16. A nucleotide amplification assay wherein food, food components, plant material or biological samples are tested for the presence of proteins or peptides which match any of the consensus sequences: (1) or (2) as defined in claim 15.

17. Method for the detection of proteins or peptides which match any of the consensus sequences (1) or (2) as defined in claim 15 in a nucleotide amplification assay in which the primer set of claim 15 is used.

18. Method for identifying cereals which are better tolerated by celiac disease patients by using a method according to claim 9.

19. Method for producing cereals which are better tolerated by celiac disease patients comprising selecting cereals having a low amount of proteins or peptides matching the consensus sequences as defined in claim 15 and crossbreeding them to produce cereals with even lower amounts of proteins and peptides.

20. Method for producing cereals which are better tolerated by celiac disease patients comprising using genetic engineering techniques to diminish the natural amount of proteins or peptides matching the consensus sequences as defined in claim 15.

21. Method according to claim 20, wherein the genetic engineering comprises transformation with recombinant DNA or induced mutation.

22. Method according to claim 21, wherein the transformation with recombinant DNA comprises homologous recombination or gene silencing.

23. Method according to claim 22 wherein the cereal is wheat.

24. A cereal plant obtained by the method of claim 22.

25. (Canceled)

26. A method to identify neo-epitopes from auto-antigens comprising:
comparing the sequence of an auto-antigen and searching for peptide sequences which match with the consensus sequences as defined in claim 15; and
screening biological material obtained from auto-immune disease patients for antibodies which bind to the peptide sequences or T cells which bind to the HLA-bound peptide sequences as identified in the previous step or peptides which are formed from said peptide sequences by deamidation of one or more Q residues.

27. A method to inhibit the binding of a T cell receptor to an HLA-bound epitope according to claim 2 comprising providing a blocking substance.

28. A method to inhibit the binding of the epitopes of claim 2 to HLA-DQ molecules comprising providing substances that block the binding of the epitopes to the HLA-DQ molecules.

29. A method to inhibit the binding of the epitopes of claim 5 to HLA molecules comprising providing substances that block the binding of the epitopes to the HLA molecules.

30. Analogue of an epitope according to claim 2 wherein the analogue is an antagonist for the activity of T cells recognising the epitope.

31. A pharmaceutical composition comprising a peptide according to claim 1.

32. A pharmaceutical composition according to claim 31 for the induction of tolerance.

33. A pharmaceutical composition according to claim 31 for the treatment of celiac disease or auto-immune disease.

34. A method to decrease sensitivity to auto-antigens by inhibiting the deamidation of said auto-antigen.

35. A method according to claim 34 wherein the inhibition comprises inhibiting the function of tTG.

36. A method according to claim 35, wherein the inhibition takes place at the site of presence of the auto-antigen.

37. (Canceled)

38. (Canceled)

39. A method to reduce the production of a peptide according to claim 3 comprising inhibiting the expression of the auto-antigen from which it is derived.

40. A tTG inhibitor/blocker comprising a peptide sequence in accordance with claim 1 and further comprises an electrophilic trap.

41. A tTG inhibitor/blocker comprising the peptide QPQLPYPQ and further comprising an electrophilic trap.

42. A pharmaceutical composition comprising a tTG inhibitor/blocker according to claim 41 or a functional equivalent or fragment thereof.

43. (Canceled)

44. A pharmaceutical composition comprising an epitope according to claim 2.
